(19) United States
(12) Patent Application Publication
(10) Pub. No.: US 2020/0333322 A1
(43) Pub. Date: Oct. 22, 2020

(54) ASSAYS WITH REDUCED INTERFERENCE

(71) Applicant: Essenlix Corporation, Monmouth Junction, NJ (US)

(72) Inventors: Stephen Y. Chou, Princeton, NJ (US); Wei Ding, Princeton, NJ (US)

(73) Assignee: Essenlix Corporation, Monmouth Junction, NJ (US)

(21) Appl. No.: 16/640,149

(22) PCT Filed: Jul. 31, 2018
(86) PCT No.: PCT/US18/44513

§ 371 Date: Feb. 19, 2020

Related U.S. Application Data
(60) Provisional application No. 62/550,349, filed on Aug. 25, 2017.
(60) Provisional application No. 62/539,508, filed on Jul. 31, 2017.

(51) Int. Cl. G01N 33/487; G01N 33/49; G01N 33/53; G01N 21/35

(52) U.S. Cl. CPC G01N 33/48785; G01N 33/49; B01L 3/5023; G01N 21/35; G01N 33/5302

(57) ABSTRACT

The present invention provides devices, systems, and methods, for performing biological and chemical assays.

### CROSS-REFERENCING

This application is a National Stage entry (§ 371) application of International Application No. PCT/US2018/044513, filed on Jul. 31, 2018, which claims the benefit of U.S. Provisional Patent Application No. 62/550,349, filed on Aug. 25, 2017, and U.S. Provisional Patent Application No. 62/539,508, filed on Jul. 31, 2017, the contents of which are relied upon and incorporated herein by reference in their entirety.

The entire disclosure of any publication or patent document mentioned herein is entirely incorporated by reference.

### FIELD

Among other things, the present invention is related to devices/apparatus and methods of performing biological and chemical assays.

### BACKGROUND

In many bio/chemical sensing and testing (e.g. immunoassay, nucleotide assay, blood cell counting, etc.), chemical reactions, and other processes, there are needs for methods and devices/apparatus that can reduce the effects from interference elements in the sample. The present invention relates to the methods, devices, apparatus, and systems that address these needs.

### BRIEF DESCRIPTION OF THE DRAWINGS

The skilled artisan will understand that the drawings, described below, are for illustration purposes only. The drawings are not intended to limit the scope of the present teachings in any way. In some Figures, the drawings are in scale. In the figures that present experimental data points, the lines that connect the data points are for guiding a viewing of the data only and have no other means.

FIG. 1 provides schematic illustrations showing some embodiments of the present invention. Panel (A) shows a detector, an imager, and a sample holder that holds a sample. Panel (B) shows an exemplary illustration of an image of the sample, demonstrating the interference element, the interference element rich regions, and the interference element poor regions.

FIG. 2 shows exemplary illustrations of the images of the sample. Panel (A) shows the sample before coagulation. Panel (B) shows the sample after coagulation.

FIG. 3 provides a schematic illustration showing some embodiments of the present invention, demonstrating the detector, the imager, and the sample holder that holds a sample, wherein the sample holder is a QMAX card (Q-card).

FIG. 4 shows an exemplary flow chart that demonstrates the process to conduct an assay that reduce the effects of interference elements.

FIG. 5 shows an exemplary embodiment of the design of a QMAX card and the basic process to measure glucose levels in a blood sample

FIG. 6 shows exemplary images of the sample, demonstrating the interference element rich region and the interference element poor region.

FIG. 7 shows a computer control system that is programmed or otherwise configured to implement methods provided herein.

### DETAILED DESCRIPTION OF EXEMPLARY EMBODIMENTS

The following detailed description illustrates some embodiments of the invention by way of example and not by way of limitation. If any, the section headings and any subtitles used herein are for organizational purposes only and are not to be construed as limiting the subject matter described in any way. The contents under a section heading and/or subtitle are not limited to the section heading and/or subtitle, but apply to the entire description of the present invention.

The citation of any publication is for its disclosure prior to the filing date and should not be construed as an admission that the present claims are not entitled to antedate such publication by virtue of prior invention. Further, the dates of publication provided can be different from the actual publication dates which can need to be independently confirmed.

It should be noted that the Figures do not intend to show the elements in strict proportion. For clarity purposes, some elements are enlarged when illustrated in the Figures. The dimensions of the elements in the Figure should be delineated from the descriptions herein provided and incorporated by reference.

### 1. Definitions

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present teachings, some exemplary methods and materials are now described.

The terms “assay” and “assaying” as used here refer to testing a sample to detect the presence and/or abundance of an analyte.

The term “analyte” as used here refers to any molecules, compounds, cells, tissues, and/or any substance that is being studied and/or analyzed. In certain embodiments, the term refers to a molecule (e.g., a protein, peptides, DNA, RNA, nucleic acid, or other molecule), cells, tissues, viruses, and nanoparticles with different shapes.

The term “interference element” as used here refers to the element in a sample that have an “interference” with a signal related to an analyte in the sample, wherein the “interference” refers to blocking, reducing, attenuating, and/or disrupting the signal related to the analyte. The cause of the interference can be physical effects, biochemical effects, or a combination of thereof. Examples of an interference element includes, but not limited to, cells, tissues, molecules, compounds, in-organic constructs, nanoparticles, air bubbles, or any combination or mixtures thereof.

The term “imager” as used here refers to a device or component of a device that includes optical parts and is configured to capture images of a samples. In some embodiments, the imager is camera. In certain embodiments, the imager is a camera that is part of a smart phone.

The term “detector” as used here refers to devices that are configured to detect and/or measure signals gathered by the detector and/or other devices/components. In some embodiments, the detector refers to a mobile device. In certain embodiments, the detector is a smart phone.

The term “software” as used here refers to a series of instructions that are configured to direct, manipulate, and/or cause a processor (e.g. a central processing unit) and associated hardware to perform specific functions, calculations, and/or operations. In some embodiments, the software is stored in and used by a computing device.

The phrase “signal related to the analyte” as used here refers to signals that is produced by a chemical, biological, and/or physical reaction that involve the analyte. In some embodiments, the signal related to the analyte allows for the detection and/or measurement of the analyte in the sample.

The term “aggregation reagent” as used here refers to any molecules, compounds, cells, tissues, and/or any substance that can induce, facilitate, strengthen, and/or accelerate the aggregation of one or more interference elements in a sample.

The term “analyte signal” refers to the signal related to the analyte. An analyte signal can be a signal directly from the analyte, a signal from a label that is attached (directly or indirectly) to the analyte, or a combination.

### 2. Working Principle

In assaying an analyte in a sample, often a signal related to the analyte is interfered by an interference element in the sample. The term “interference element” as used here refers to the element in a sample that have an “interference” with a signal related to an analyte in the sample, wherein the “interference” refers to blocking, reducing, attenuating, and/or disrupting the signal related to the analyte. The cause of the interference can be physical effects, biochemical effects, or a combination thereof. Examples of an interference element include, but are not limited to: cells, tissues, molecules, compounds, in-organic constructs, nanoparticles, air bubbles, or any combination or mixtures thereof.

One aspect of the present invention provides devices/apparatus and methods that can reduce the interference.

### Reduce Interference

PI-1 For a sample that contains an analyte and interference element(s), the present invention reduces an interference of the interference elements to a signal related to analyte by a method comprising:

(a) having the sample that contains an analyte and one or more interference elements, where the interference elements are aggregated into a region(s) of the sample, that makes the interference element concentration in the region(s) (“interference element rich region”) substantially higher than that in other regions of the sample (“interference element poor region”);

(b) identifying the interference element poor region(s) in at least a part of the sample; and

(c) measuring the signal related to the analyte (“analyte signal”) in the interference element poor region(s).

In some embodiments of the method of PI-1, the identifying of the IE poor region(s) can be achieved by imaging and image analysis (e.g. using software) of the at least part of the sample.

In some embodiments of the method of PI-1, the step (b) and (c) are performed at the same time.

In some embodiments of the method of PI-1, the step (b) and (c) are performed at different same time.

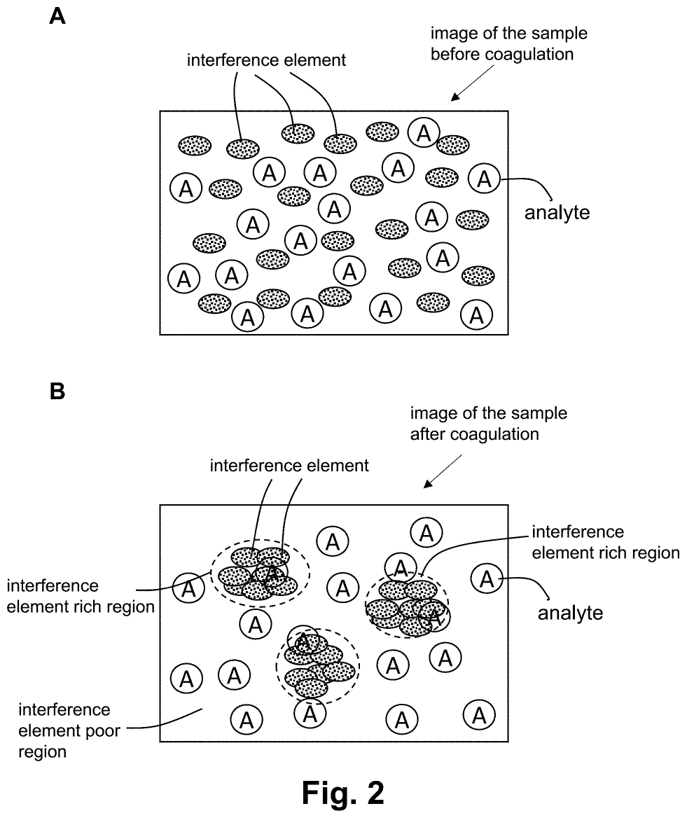

In certain embodiments, as illustrated in FIG. 2, initially the interference elements are substantially statistically uniform distributed in the sample, but later the interference elements (IE) become aggregated into IE rich region and IE poor region. The aggregation can happen (a) naturally without using any additional aggregation reagent(s), or (a) by adding aggregation reagent(s) into the sample.

One example of aggregation naturally is a whole blood sample, where the red blood cells and platelets in a fresh blood out of human body will naturally aggregate (if there is no anti-aggregation agent being added).

In some embodiment, it further comprises a step of identifying the interference element (IE) rich region and measuring the signal related to the analyte in the IE rich region.

### Microdomains

In some embodiments, the interference element poor and/or rich regions in a sample are microdomains. Herein the term “microdomain” means that each interference element poor and/or rich region has an average dimension of 800 micron or less.

In certain embodiments, only the interference element poor regions or only the interference element high regions are microdomain. In certain embodiments, both interference element poor regions and interference element high regions are microdomain, and interference element poor and rich regions are intermixed together.

The apparatus, kit, or method of any prior embodiments, wherein the interference element poor and/or rich regions in the sample form one or more microdomains, and wherein a microdomain is an interference element poor or region that has an average dimension of 800 um or less.

In some embodiments, each of the microdomain has an average dimension of less than 1 um, 10 um, 50 um, 100 um, 200 um, 250 um, 500 um, 600 um, 700 um, or 800 um, or in a range between any of the two values. In certain embodiments, each of the one or more microdomain has an average dimension of 700 um or less. In certain embodiments, each of the one or more microdomain has an average dimension of 600 um or less. In certain embodiments, each of the one or more microdomain has an average dimension of 500 um or less. In certain embodiments, each of the one or more microdomain has an average dimension of 250 um or less. In certain embodiments, each of the one or more microdomain has an average dimension of 100 um or less. In certain embodiments, each of the one or more microdomain has an average dimension of 50 um or less. In certain embodiments, each of the one or more microdomain has an average dimension of 10 um or less. In certain embodiments, each of the one or more microdomain has an average dimension of 1 um or less.

In some embodiments, each of the one or more microdomain has an average dimension in the range of 1-800 um, 50-800 um, 100-800 um, 250-800 um, 500-800 um, or 600-800 um. In certain embodiments, each of the one or more microdomain has an average dimension in the range of 1-800 um, 1-700 um, 1-600 um, 1-500 um, 1-250 um, 1-100 um, 1-50 um, 1-25 um, or 1-10 um.

### Analyte Concentration Determination

According to the present invention, the concentration of analytes in the sample by measuring the signal related to the analyte in an IE poor region and measuring the volume of this particular IE poor region.

According to the present invention, the concentration of analytes in the sample by measuring the signal related to the analyte in several IE poor regions and measuring the volume of the several IE poor regions.

According to the present invention, in some embodiments for measuring the volume of one or several IE poor regions, the area and the height of the IE poor regions are measured.

According to the present invention, in some embodiments for measuring the volume of one or several IE poor regions, a sample is sandwiched between two plates that make a sample with a uniform thickness, and the thickness and the area of the IE poor region is measured.

According to the present invention, in some embodiments for measuring the volume of one or several IE poor regions, a sample is sandwiched between two plates that make a sample with a uniform thickness, wherein the sample thickness is predetermined by spacers placed on the surface of one or both plates while the area of the area of the IE poor region is measured.

According to the present invention, in some embodiments, the measuring of the area of the IE poor region is by a camera.

In some embodiments, the sample has a uniform thickness of 500 nm or less, 1000 nm or less, 2 μm (micron) or less, 5 μm or less, 10 μm or less, 20 μm or less, 50 μm or less, 100 μm or less, 150 μm or less, 200 μm or less, 300 μm or less, 500 μm or less, 800 μm or less, 1 mm (millimeter) or less, 2 mm or less, 3 mm or less, 5 mm or less, 10 mm or less, or in a range between any two of these values. In some embodiments, the sample has lateral size of 0.1 mm² or less, 0.2 mm² or less, 0.5 mm² or less, 1 mm² or less, 2 mm² or less, 5 mm² or less, 10 mm² or less, 20 mm² or less, 50 mm² or less, 100 mm² or less, 200 mm² or less, 500 mm² or less, 1000 mm² or less, 2000 mm² or less, 5000 mm² or less, or 10000 mm² or less, or in a range between any of the two values. In some embodiments, the thickness of the sample is regulated by spacers in the sample holder (e.g. a QMAX device); in certain embodiments, the thickness of the sample is the same as the height of the spacers. The lateral area of the sample can be calculated by the area captured by the imager and level of magnification. In some embodiments, a working curve with known analyte concentration and corresponding signal intensity can be used to determine the concentration of the analyte in the sample, based on the signal intensity from the sample, or the signal intensity from the interference element poor region(s) of the sample.

The device and methods use imager and software that are configured to identify (a) the regions in the sample that are occupied by the one or more inference elements (“interference element rich region”), and/or (b) the regions in the sample that are not occupied by the one or more inference elements (“interference element poor region”). The “virtual separation” by the imager and software between the interference element rich region and the interference element poor region facilitates the reduction of interference from the interference elements for the analysis of signals related to the analyte.

Another aspect of the present invention provides devices/apparatus and methods for improving the elimination of interference from interference elements during sample analysis, where the sample comprises or is supplied with an aggregation agent that is configured to induce aggregation of the interference elements. The aggregation of the interference elements helps limit the geographical distribution of the interference elements, thereby facilitating the reduction of interference from the interference elements.

Yet another aspect of the present invention provides devices/apparatus and methods for reducing and/or eliminating interference from interference elements during sample analysis, where the sample is compressed and confined by the two plates into a thin sample layer. The reduced thickness of the sample increases the speed of the sample analysis and facilitates the separation between the interference element rich regions and the interference element poor regions.

Still another aspect of the present invention provides devices/apparatus and methods for reducing and/or eliminating interference from interference elements by identifying and distinguishing interference element rich regions and interference element poor regions. In some embodiments, the device/apparatus and methods herein disclosed focus on signals related to the analyte in the interference element poor regions and use such signals as a better reflection of the presence and/or concentration of the analyte in the sample.

### 3. Exemplary Embodiments

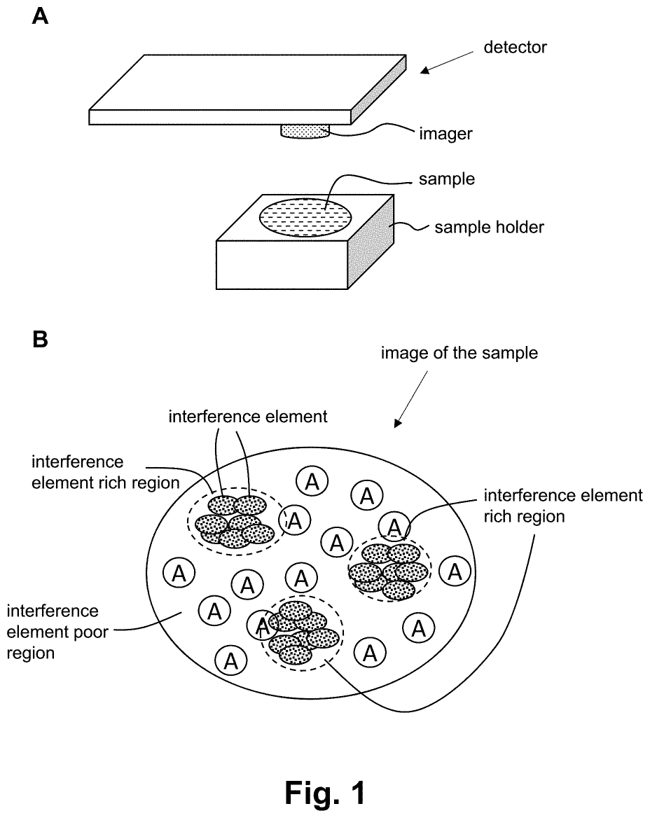

FIG. 1 provides schematic illustrations showing some embodiments of the present invention. Panel (A) shows an apparatus that comprises a detector, an imager, and a sample holder that holds a sample. Panel (B) shows an exemplary illustration of an image of the sample taken by the apparatus depicted in panel (A), demonstrating the interference elements, the interference element rich regions, and the interference element poor region. In FIGS. 1 and 2, the “A” in the circle refers to analyte and demonstrates the distribution of the analyte.

As shown in FIG. 1, panel (A), the apparatus comprises a detector, an imager, and a sample holder that holds a sample. In some embodiments, the imager is an independent device from the detector but connected to the detector. In some embodiments, the imager an independent device from the detector but not connected to the detector. In some embodiments, the imager is part of the detector but is not structurally integrated in a main detector body. In some embodiments, the imager is part of the detector and integrated in the main detector body. In some embodiments, the imager comprises a camera. In some embodiments, the detector comprises a mobile device. In certain embodiments, the detector is a smart phone. In certain embodiments, the imager is a camera integrated in the smart phone.

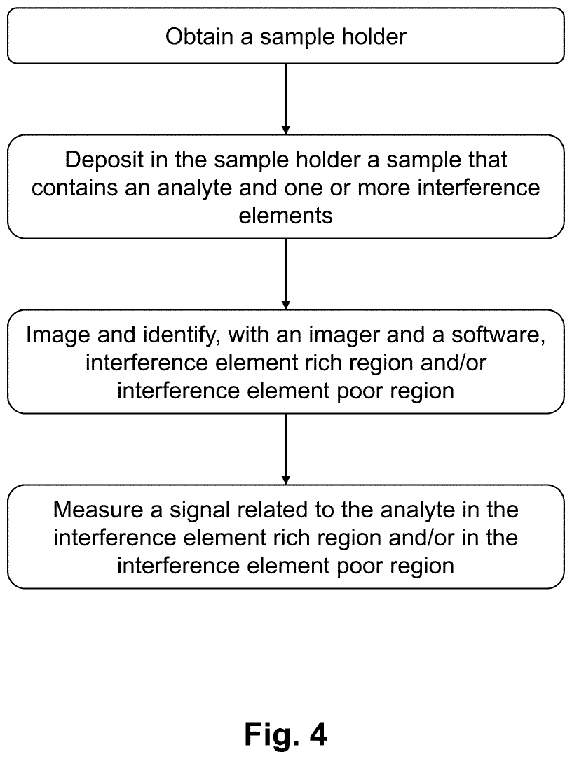

FIG. 4 shows an exemplary flow chart that demonstrates the process to conduct an assay that reduce the effects of interference elements. As shown in FIG. 4, in some embodiments, the method comprises:

i. obtaining a sample holder;

ii. depositing in the sample holder a sample that contains an analyte and one or more interference elements;

iii. imaging and identifying, with an imager and a software, (a) the regions in the sample that are occupied by the one or more has less inference elements concentration (“interference element rich region”) and/or (b) than another regions in the sample that are not occupied by the one or more inference elements (“interference element free poor region”), and/or (b) an interference element rich region; and

iv. measuring a signal related to the analyte in the interference element rich region and/or in the interference element free poor region.

In some embodiments, the signal related to analyte in the interference element poor region is measured. In some embodiments, the method further comprises calculating a concentration of the analyte in the sample based on the signal related to the analyte in the interference element rich region and/or in the interference element poor region. In some embodiments, the method further comprises calculating a concentration of the analyte in the sample based on the signal related to the analyte in the interference element poor region.

### Example-1. Interference Removal Assay (IRA) to Test Glucose Level in Fresh Blood

Here we describe an experiment of interference removal assay (IRA) for testing glucose level in fresh blood according to one embodiment of the present invention.

In this experiment, the device comprises a first plate and a second plate. The first plate has a size of 24 mm×32 mm, a thickness of 1 mm, made of white non-transparent polystyrene with a surface roughness around 5 um. The second plate has a size of 22 mm×25 mm, a thickness of 0.175 mm, made of transparent PMMA with a pillar array on top of it. The pillar array has 30×40 um pillar size, 80 um inter pillar distance, and 10 um pillar height.

The second plate was coated with a glucose colorimetric reagent array. The glucose colorimetric reagent contains GO enzyme with 200 u/mL, HRP enzyme with 200 u/mL, 4-AAP with 20 mM, TOOS with 20 mM in pure water. The colorimetric reagent array has a size of 20 mm by 20 mm, printed by typical liquid dot printing machine. Each droplet in the array has a volume of 2.5 nL with a period of 500 um. Then the second plate was air dried for 5 min in the dark chamber.

The reader for IRA is built on iphone 6s, with an imaging lens (with a focus distance 4 mm) before the camera and a side emitting fiber before the iphone LED to create a uniform lighting onto the device underneath it.

The sample for testing is the fresh blood without any anticoagulant with a glucose concentration of 24 mM, 15 mM, 9 mM, 6.5 mM and 4 mM.

The experiment was conducted according to the following procedures:

### 1. Ira QMAX Assay.

2 μL fresh blood with different glucose level as 24 mM, 15 mM, 9 mM, 6.5 mM and 4 mM were dropped in the center of the first plates. The second plates were immediately pressed by hand onto the first plates with colorimetric reagents side facing the blood. The device was incubated for 3 min at room temperature before the testing.

### 2. Imaging.

Without any washing, the bright field images of device were taken by above iphone 6s setup. FIG. E2 shows exemplary pictures of the bright field with the IRA QMAX device to test fresh blood with glucose level of 24 mM, 15 mM, 9 mM, 6.5 mM and 4 mM.

As shown in the figure, we found that, in this exemplary experiment, the bright field images of device have two clear separated regions. As marked in the figure, one region is blood cell aggregation region with relative dark color. The other region is plasma region without any cells.

### 3. Analyzing.

The software separates the plasma region from the blood cell aggregation region, and average the color intensity of the plasma region. The glucose level of the blood is directly correlated to the average color intensity in the plasma region.

### 4. Sample Holder

In some embodiments, the sample holder comprises wells that are configured to hold the sample. The depth of each well can be 500 nm or less, 1000 nm or less, 2 μm (micron) or less, 5 μm or less, 10 μm or less, 20 μm or less, 50 μm or less, 100 μm or less, 150 μm or less, 200 μm or less, 300 μm or less, 500 μm or less, 800 μm or less, 1 mm (millimeter) or less, 2 mm or less, 3 mm or less, 5 mm or less, 10 mm or less, or in a range between any two of these values. The width of each well can be 1 um or less, 2 μm or less, 5 μm or less, 10 μm or less, 20 μm or less, 50 μm or less, 100 μm or less, 150 μm or less, 200 μm or less, 300 μm or less, 500 μm or less, 800 μm or less, 1 mm (millimeter) or less, 2 mm or less, 3 mm or less, 5 mm or less, 10 mm or less, or in a range between any two of these values.

In some embodiments, the sample holder comprises a first plate, a second plate, and spacers, wherein the spacers are configured to regulate a gap between the plates when the plates are pressed against each, compressing the sample into a thin layer. In certain embodiments, the sample holder is a QMAX device (or CROF device) as described in PCT/US2016/051775 filed on Sep. 14, 2016, which is incorporated by reference by its entirety for all purposes.

In some embodiments, the sample holder comprises a QMAX card (Q-card), which comprises a first plate, a second plate, and spacers, wherein the spacers are configured to regulate a gap between the plates when the plates are pressed against each, compressing the sample into a thin layer. In certain embodiments, the first plate and the second plate of the Q-card are connected by a hinge, which allows the two plates to pivot against each other.

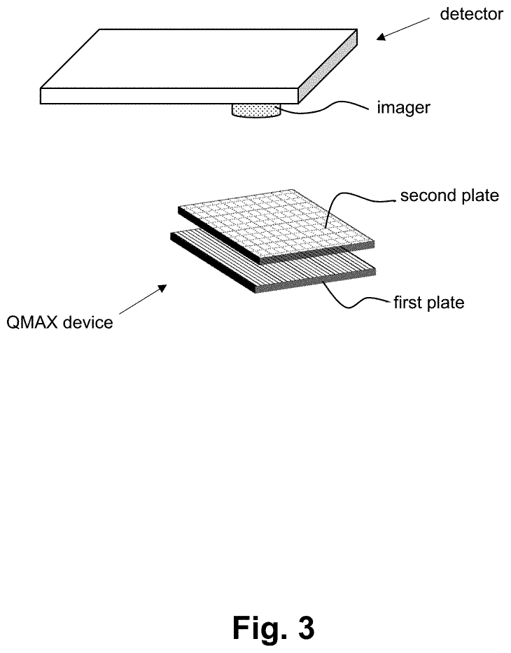

FIG. 3 provides a schematic illustration showing some embodiments of the present invention, demonstrating the detector, the imager, and the sample holder that holds a sample, wherein the sample holder is a QMAX card (Q-card, Q: quantification; M: magnifying; A: adding reagents; X: acceleration; also known as compressed regulated open flow (CROF) device).

As shown in FIG. 3, the Q-card comprises a first plate and a second plate, and the two plates are relatively movable to each other into different configurations, including an open configuration and a closed configuration. The closed configuration is depicted in the figure, where the sample (not shown) is compressed by the two plates into a thin layer. In some embodiments, the thickness of the thin layer is 100 nm or less, 500 nm or less, 1 μm (micron) or less, 2 μm or less, 5 μm or less, 10 μm or less, 20 μm or less, 50 μm or less, 100 μm or less, 200 μm or less, 500 μm or less, 1 mm or less, 2 mm or less, 5 mm or less, 10 mm or less, or in a range between any two of these values. In preferred embodiments, the thickness of the thin layer is 1 μm or less, 2 μm or less, 5 μm or less, 10 μm or less, 20 μm or less, 50 μm or less, 100 μm or less, or in a range between any two of these values.

### 5. Sample Thickness

Limiting the sample thickness can offer an advantage of reducing the effect of the interference elements, even when the interference elements have been aggregated into interference element rich (IER) and interference element poor (IEP) regions. This is because that for a thicker sample, the IER regions and IEP regions can overlap in the thickness direction.

To detect a signal from only a region of interference element poor or rich, it is highly desirable to reduce or eliminate the spatial overlap between the IE poor and rich regions in the direction of the sample thickness. One way to reduce and eliminate the overlap is to use a thin sample thickness. One way to achieve a thin sample thickness is to use two plates to confine a sample into a thin thickness, wherein the two plates can be (a) fixed wherein the sample gets into the space between the plates by flow, and (b) movable relative to each other, wherein the sample can be compressed into a thin layer.

In some embodiments, the sample has a uniform thickness of 500 nm or less, 1000 nm or less, 2 μm (micron) or less, 5 μm or less, 10 μm or less, 20 μm or less, 50 μm or less, 100 μm or less, 150 μm or less, 200 μm or less, 300 μm or less, 500 μm or less, 800 μm or less, 1 mm (millimeter) or less, 2 mm or less, 3 mm or less, 5 mm or less, 10 mm or less, or in a range between any two of these values.

In certain preferred embodiments, the sample has a uniform thickness of 500 nm or less, 1000 nm or less, 2 μm (micron) or less, 5 μm or less, 10 μm or less, 20 μm or less, 50 μm or less, 100 μm or less, or in a range between any two of these values.

In certain preferred embodiments, the sample has a uniform thickness of 500 nm or less, 1000 nm or less, 2 μm (micron) or less, 5 μm or less, 10 μm or less, 20 μm or less, or in a range between any two of these values.

In some embodiments, at least part of the sample is compressed into a thin layer, and for a specific part of the sample that has an average thickness of 500 um or less, 400 um or less, 300 um or less, 200 um or less, 175 um or less, 150 um or less, 125 um or less, 100 um or less, 75 um or less, 50 um or less, 40 um or less, 30 um or less, 20 um or less, 10 um or less, 5 um or less, 4 um or less, 3 um or less, 2 um or less, 1.8 um or less, 1.5 um or less, 1 um or less, 0.5 um or less, 0.2 um or less, 0.1 um or less, 50 nm or less, 20 nm or less, 10 nm or less, or in a range between any of the two values, only the interference rich regions exist.

In some embodiments, at least part of the sample is compressed into a thin layer, and for a specific part of the sample that has an average thickness of 500 um or less, 400 um or less, 300 um or less, 200 um or less, 175 um or less, 150 um or less, 125 um or less, 100 um or less, 75 um or less, 50 um or less, 40 um or less, 30 um or less, 20 um or less, 10 um or less, 5 um or less, 4 um or less, 3 um or less, 2 um or less, 1.8 um or less, 1.5 um or less, 1 um or less, 0.5 um or less, 0.2 um or less, 0.1 um or less, 50 nm or less, 20 nm or less, 10 nm or less, or in a range between any of the two values, only the interference poor regions exist.

In some embodiments, at least part of the sample is compressed into a thin layer that has an average thickness of in a range of 0.5-2 um, 0.5-3 um, 0.5-5 um, 0.5-10 um, 0.5-20 um, 0.5-30 um, or 0.5-50 um.

In certain embodiments, at least part of the sample is compressed into a thin layer that has an average thickness of 500 um or less, 200 um or less, 100 um or less, 50 um or less, 25 um or less, 10 um or less, 5 um or less, 3 um or less, 2 um or less, 1 um or less, 500 nm or less, 100 nm or less, or in range between any of the two values.

In certain embodiments, at least part of the sample is compressed into a thin layer that has an average thickness in the range of 0.5-2 um, 0.5-3 um, or 0.5-5 um. In certain embodiments, the average thickness of the layer of uniform thickness is in the range of 2 um to 2.2 um and the sample is blood. In certain embodiments, the average thickness of the layer of uniform thickness is in the range of 2.2 um to 2.6 um and the sample is blood. In certain embodiments, the average thickness of the layer of uniform thickness is in the range of 1.8 um to 2 um and the sample is blood. In certain embodiments, the average thickness of the layer of uniform thickness is in the range of 2.6 um to 3.8 um and the sample is blood. In certain embodiments, the average thickness of the layer of uniform thickness is in the range of 1.8 um to 3.8 um and the sample is whole blood without a dilution by another liquid.

In some embodiments, the average thickness of the layer of uniform thickness is about equal to a minimum dimension of an analyte in the sample.

In some embodiments, the final sample thickness device is configured to analyze the sample in 300 seconds or less. In certain embodiments, the final sample thickness device is configured to analyze the sample in 180 seconds or less. In certain embodiments, the final sample thickness device is configured to analyze the sample in 60 seconds or less. In certain embodiments, the final sample thickness device is configured to analyze the sample in 30 seconds or less.

### 6. IE Rich Region and IE Poor Region

The IE, the IE rich region, and the IE poor region can be of any scale, such as but not limited to the nano-meter scale, micrometer scale, or millimeter scale. For example, in some embodiments, the IE rich region and/or the IE poor region has an lateral dimension of less than 10 nm, 50 nm, 100 nm, 500 um, 1 um, 5 um, 10 um, 50 um, 100 um, 500 um, 1 mm, 5 mm, 10 mm, 50 mm, 100 mm, or 500 mm, or in a range between any of the values.

In some embodiments, an IE rich region is defined as a region that has at least 50%, 60%, 70%, 80%, 90%, or 95% of the lateral area being covered by the IE, where the IE are substantially connected. A substantially IE region is defined as a region that has at least 70%, 80%, 90%, or 95% of the lateral area being covered by the IE.

In some embodiments, an IE poor region is defined as a region that has at most 50%, 40%, 30%, 20%, 10%, or 5% of the lateral area being covered by the IE. A substantially IE poor region is defined as a region that has at most 30%, 20%, 10%, or 5% of the lateral area being covered by the IE.

In some embodiments, the IE rich region and the IE poor region are defined by the relative concentration of the IE in these regions. In certain embodiments, the ratio of IE concentration in the IE rich region to the IE concentration in the IE poor region is equal to or more than 10000:1, equal to or more than 1000:1, equal to or more than 500:1, equal to or more than 100:1, equal to or more than 50:1, equal to or more than 20:1, equal to or more than 10:1, equal to or more than 5:1, or equal to or more than 2:1, or in a range between any of the two values.

### 7. Samples and Analytes

In some embodiments, the analyte to be detected in the homogeneous assay includes, but not limited to, cells, viruses, proteins, peptides, DNAs, RNAs, oligonucleotides, and any combination thereof.

In some embodiments, the present invention finds use in detecting biomarkers for a disease or disease state. In certain instances, the present invention finds use in detecting biomarkers for the characterization of cell signaling pathways and intracellular communication for drug discovery and vaccine development. For example, the present invention may be used to detect and/or quantify the amount of biomarkers in diseased, healthy or benign samples. In certain embodiments, the present invention finds use in detecting biomarkers for an infectious disease or disease state. In some cases, the biomarkers can be molecular biomarkers, such as but not limited to proteins, nucleic acids, carbohydrates, small molecules, and the like. The present invention find use in diagnostic assays, such as, but not limited to, the following: detecting and/or quantifying biomarkers, as described above; screening assays, where samples are tested at regular intervals for asymptomatic subjects; prognostic assays, where the presence and or quantity of a biomarker is used to predict a likely disease course; stratification assays, where a subject's response to different drug treatments can be predicted; efficacy assays, where the efficacy of a drug treatment is monitored; and the like.

The present invention has applications in (a) the detection, purification and quantification of chemical compounds or biomolecules that correlates with the stage of certain diseases, e.g., infectious and parasitic disease, injuries, cardiovascular disease, cancer, mental disorders, neuropsychiatric disorders and organic diseases, e.g., pulmonary diseases, renal diseases, (b) the detection, purification and quantification of microorganism, e.g., virus, fungus and bacteria from environment, e.g., water, soil, or biological samples, e.g., tissues, bodily fluids, (c) the detection, quantification of chemical compounds or biological samples that pose hazard to food safety or national security, e.g. toxic waste, anthrax, (d) quantification of vital parameters in medical or physiological monitor, e.g., glucose, blood oxygen level, total blood count, (e) the detection and quantification of specific DNA or RNA from biosamples, e.g., cells, viruses, bodily fluids, (f) the sequencing and comparing of genetic sequences in DNA in the chromosomes and mitochondria for genome analysis or (g) to detect reaction products, e.g., during synthesis or purification of pharmaceuticals.

In some embodiments, the liquid sample is made from a biological sample selected from the group consisting of: amniotic fluid, aqueous humour, vitreous humour, blood (e.g., whole blood, fractionated blood, plasma or serum), breast milk, cerebrospinal fluid (CSF), cerumen (earwax), chyle, chime, endolymph, perilymph, feces, breath, gastric acid, gastric juice, lymph, mucus (including nasal drainage and phlegm), pericardial fluid, peritoneal fluid, pleural fluid, pus, rheum, saliva, exhaled breath condensates, sebum, semen, sputum, sweat, synovial fluid, tears, vomit, urine, and any combination thereof.

In some embodiments, the sample is an environmental liquid sample from a source selected from the group consisting of: river, lake, pond, ocean, glaciers, icebergs, rain, snow, sewage, reservoirs, tap water, or drinking water, solid samples from soil, compost, sand, rocks, concrete, wood, brick, sewage, and any combination thereof.

In some embodiments, the sample is an environmental gaseous sample from a source selected from the group consisting of the air, underwater heat vents, industrial exhaust, vehicular exhaust, and any combination thereof.

In some embodiments, the sample is a foodstuff sample selected from the group consisting of raw ingredients, cooked food, plant and animal sources of food, preprocessed food, and partially or fully processed food, and any combination thereof.

### 8. Assay Types and Signals

The device, apparatus, kit, and method of the present invention can be used for various types of assays, including but limited to immunoassays, immunochemistry assays, immunohistochemistry assays, immunocytochemistry assays, immunoblotting assays, immunoprecipitation assays, nucleic acid assays, nucleic acid hybridization assays, northern blotting assays, southern blotting assays, DNA footprinting assays, microarrays, nucleic acid sequencing, polymerase chain reaction (PCR) assays, ligation assays, cloning assays, nephelometry assays, and cell aggregation assays, and any variations or combinations thereof.

In some embodiments, the assay is a sandwich assay, in which capture agent and detection agent are configured to bind to analyte at different locations thereof, forming capture agent-analyte-detection agent sandwich.

In some embodiments, the assay is a competitive assay, in which analyte and detection agent compete with each other to bind to the capture agent.

In some embodiments, the assay is a nephelometry assay that is used to determine the levels of several blood plasma proteins, such as but not limited to immunoglobulin M, immunoglobulin G, and/or immunoglobulin A.

In some embodiments, the assay is an immunoassay, in which protein analyte is detected by antibody-antigen interaction. In some embodiments, the assay is a nucleic acid assay, in which nucleic acids (e.g. DNA or RNA) are detected by hybridization with complementary oligonucleotide probes.

In some embodiments, the assay utilizes light signals as readout. In some embodiments, the assay utilizes magnetic signals as readout. In some embodiments, the assay utilizes electric signals as readout. In some embodiments, the assay utilizes signals in any other form as readout.

In some embodiments, the light signal from the assay is luminescence selected from photoluminescence, electroluminescence, and electrochemiluminescence. In some embodiments, the light signal is light absorption, reflection, transmission, diffraction, scattering, or diffusion. In some embodiments, the light signal is surface Raman scattering. In some embodiments, the electrical signal is electrical impedance selected from resistance, capacitance, and inductance. In some embodiments, the magnetic signal is magnetic relaxivity. In some embodiments, the signal is any combination of the foregoing signal forms.

It is another aspect of the present invention to provide devices and methods with multiplexing capability for homogeneous assays.

In some embodiments, the sample comprises more than one analyte of interest, and there is need to detect the more than one analytes simultaneously using the same device (“multiplexing”).

In addition to immunoassays, the present invention also finds use in homogeneous nucleic acid hybridization assays.

In some embodiments, in nucleic acid hybridization assays, the capture agent is oligonucleotide or oligomimetics capture probe. In some embodiments of the present invention, the concentration surface, protrusions, or beads are coated with the capture probes. The capture probes are complementary to one part of the nucleic acid analyte, therefore capturing the analyte to the surface. Further, the analyte is bound with a labeled detection probe that is complementary to another part of the analyte.

One aspect of the present invention provides a device for homogeneous assay with concentration beads. In some embodiments, the device comprises: a first plate, a second plate, and spacers. In some embodiments, the plates are movable relative to each other into different configurations, including an open configuration and a closed configuration. In some embodiments, each of the plates has, on its respective inner surface, a sample contact area for contacting a sample suspected of comprising an analyte. In some embodiments, one or both of the plates comprise the spacers, at least one of the spacers is inside the sample contact area, and the spacers have a predetermined substantially uniform height. In some embodiments, one or both of the plates comprise, on the respective inner surface, a plurality of beads that have capture agent immobilized thereon, wherein the capture agent is capable of binding to and immobilizing the analyte. In some embodiments, one or both of the plates comprise, on the respective inner surface, detection agent that is configured to, upon contacting the sample, be dissolved in the sample and bind to the analyte.

In some embodiments, the device, apparatus, and method of the present invention can be used to create a homogenous assay by using a surface amplification layer. In certain embodiments, the sample can be applied to the layer and bind to the layer; in certain embodiments, interference element (IE) aggregation ensues. In some embodiments, an imager and a detector, with the associated software, can be used to measure and analyze signals in IE poor region. In some embodiments, the amplification layer can be a Dots-on-Pillar Antenna-Array (D2PA). Some embodiments of the amplification layer are disclosed and/or described in U.S. Pat. Nos. 9,182,338 and 9,013,690, which are incorporated in their entireties for all purposes.

### 9. Aggregation Agent

In certain embodiments, the aggregation reagent includes but is not limited to fibrinogen (and subunits thereof), thrombin and prothrombin, certain polymers, certain dextran fractions (e.g. Dx-500, Dx-100, and Dx-70), poly(ethylene glycol), or polyvinylprrolidone (PVP, e.g. PVP-360 and PVP-40), or any combination thereof.

### 10. Applications

The present invention has applications in (a) the detection, purification and quantification of chemical compounds or biomolecules that correlates with the stage of certain diseases, e.g., infectious and parasitic disease, injuries, cardiovascular disease, cancer, mental disorders, neuropsychiatric disorders and organic diseases, e.g., pulmonary diseases, renal diseases, (b) the detection, purification and quantification of microorganism, e.g., virus, fungus and bacteria from environment, e.g., water, soil, or biological samples, e.g., tissues, bodily fluids, (c) the detection, quantification of chemical compounds or biological samples that pose hazard to food safety or national security, e.g. toxic waste, anthrax, (d) quantification of vital parameters in medical or physiological monitor, e.g., glucose, blood oxygen level, total blood count, (e) the detection and quantification of specific DNA or RNA from biosamples, e.g., cells, viruses, bodily fluids, (f) the sequencing and comparing of genetic sequences in DNA in the chromosomes and mitochondria for genome analysis or (g) to detect reaction products, e.g., during synthesis or purification of pharmaceuticals.

In some embodiments, the liquid sample is made from a biological sample selected from the group consisting of: amniotic fluid, aqueous humour, vitreous humour, blood (e.g., whole blood, fractionated blood, plasma or serum), breast milk, cerebrospinal fluid (CSF), cerumen (earwax), chyle, chime, endolymph, perilymph, feces, breath, gastric acid, gastric juice, lymph, mucus (including nasal drainage and phlegm), pericardial fluid, peritoneal fluid, pleural fluid, pus, rheum, saliva, exhaled breath condensates, sebum, semen, sputum, sweat, synovial fluid, tears, vomit, urine, and any combination thereof.

In some embodiments, the sample is an environmental liquid sample from a source selected from the group consisting of: river, lake, pond, ocean, glaciers, icebergs, rain, snow, sewage, reservoirs, tap water, or drinking water, solid samples from soil, compost, sand, rocks, concrete, wood, brick, sewage, and any combination thereof.

In some embodiments, the sample is an environmental gaseous sample from a source selected from the group consisting of: the air, underwater heat vents, industrial exhaust, vehicular exhaust, and any combination thereof.

In some embodiments, the sample is a foodstuff sample selected from the group consisting of raw ingredients, cooked food, plant and animal sources of food, preprocessed food, and partially or fully processed food, and any combination thereof.

### EXAMPLES

Panel (B) of FIG. 1 shows an exemplary illustration of an image of the sample taken by the apparatus depicted in panel (A), demonstrating the interference elements, the interference element rich regions, and the interference element poor region.

In some embodiments, the interference elements can interfere with the signals from the sample if no additional steps are taken. The interference elements can be cells, tissues, molecules, compounds, in-organic constructs (e.g. dust or air bubble), or any combination or mixtures thereof. In some embodiments, the interference elements are present in large quantities in the sample. In some embodiments, the interference elements are sparse and/or scattered. In some embodiments, the interference elements block, reduce, attenuate, disrupt and/or cover the signal from the analyte. In certain embodiments, the interference from the interference elements are due to the physical, chemical, and/or biological properties of the interference elements. Whether a specific entity in a sample is a considered a interference element also depends the nature and purpose of the assay. For example, in certain assays, the red blood cells in a blood sample is considered an interference element that interferes with the detection and/or measurement of the signals from an analyte in the plasma, or in the white blood cells. However, in certain assays when the analyte is associated with (or is) the red blood cells, the red blood cells are not considered an interference element.

In some embodiments, the interference elements in the sample can be distinguished from the rest of the sample. In some embodiments, the interference elements aggregate after the sample is deposited in the sample holder, facilitating the distinction of the interference elements. In some embodiments, a biological/chemical reaction occurs during and/or after the sample is deposited. In some embodiments, the biological/chemical reaction results in showing of color and/or generation of signals. In certain embodiments, the reaction is a colorimetric reaction and the sample shows a specific color with the procession of the reaction. In certain embodiments, the reaction a fluorescence reaction and the sample provide a fluorescent signal when stimulated. In some embodiments, the presence of the interference elements interferes with the detection and/or measurement of the signals from the reaction.

As shown in panel (B) of FIG. 1, in some embodiments, the sample has interference element rich regions and interference element poor regions. In certain embodiments, the interference elements aggregate; in certain embodiments, the interference elements do not aggregate. While aggregation can facilitate identifying and distinguishing the interference element rich and interference poor regions, aggregation is not a requirement for the devices/apparatus and methods herein disclosed.

In some embodiments, the interference element rich region has an area that is at least 30%, 40%, 50%, 60%, 70%, 80%, 90%, or 99% covered by the interference elements. In certain embodiments, the interference element rich region has an area that is at least 70%, 75%, 80%, 85%, 90%, 95%, or 99% covered by the interference elements.

In some embodiments, the interference element poor region has an area that is less than 50%, 40%, 30%, 20%, 10%, 5%, 1%, or 0.1% covered by the interference elements. In some embodiments, the interference element poor region has an area that is less than 25%, 20%, 15%, 10%, 5%, 1%, or 0.1% covered by the interference elements.

In some embodiments, there is a single interference element rich region in the sample. In some embodiments, there is a single interference element poor region in the sample.

In certain embodiments, the imager is configured to identify and distinguish the interference element rich region and the interference element poor region. In certain embodiments, with the aggregation of the interference elements, it is easier for the imager to identify and distinguish the interference element rich region and the interference element poor region as compared to the sample in which there is no aggregation of the interference element. The imager can be associated with a software, which includes a series of instructions that can direct the imager and/or its associated structures to perform certain actions.

In some embodiments, the detector is configured to detect a signal related to the analyte in the interference element poor region. In some embodiments, the detector is configured to detect a signal related to the analyte in the interference element rich region. In some embodiments, the detector is configured to detect a signal related to the analyte in both regions. As discussed above, in some embodiments, the imager is part of the detector. With the aid of the separation of the interference element poor and rich regions, the detector comprises hardware and software that are configured to: 1) distinguish the signal emanating from the interference element poor and rich regions with the capability of reading and analyzing the signal emanating from both regions; or 2) read and analyze the signal emanating from either the interference element poor or rich region alone.

In some embodiments, the detector is directed to calculate the concentration of the analyte in the sample. In certain embodiments, the sample is compressed into a thin layer with measurable thickness. In certain embodiments, the sample is compressed into a layer with uniform thickness (e.g. by the Q-card). For example, in some embodiments, the Q-card comprises spacers that determine the gap between the plates when the plates are pressed against each other, thus determining the thickness of the sample when the sample is between the plates. When the thickness of the sample is available, in certain embodiments, the volume of the sample (or part of the sample) can be determined by measuring the relevant area (e.g. the entire sample area, the interference element rich region, and/or the interference element poor region).

In some embodiments, the sample is original, diluted, or processed forms of: bodily fluids, stool, amniotic fluid, aqueous humour, vitreous humour, blood, whole blood, fractionated blood, plasma, serum, breast milk, cerebrospinal fluid, cerumen, chyle, chime, endolymph, perilymph, feces, gastric acid, gastric juice, lymph, mucus, nasal drainage, phlegm, pericardial fluid, peritoneal fluid, pleural fluid, pus, rheum, saliva, sebum, semen, sputum, sweat, synovial fluid, tears, vomit, urine, or exhaled breath condensate. In some embodiments, the sample is original, diluted, or processed forms of blood. In certain embodiments, the sample comprises whole blood.

In some embodiments, the sample comprises an aggregation agent that induces aggregation of the interference elements. In certain embodiments, the aggregation agent comprises: fibrinogen (and subunits thereof), thrombin and prothrombin, certain dextran fractions (e.g. Dx-500, Dx-100, and Dx-70), poly(ethylene glycol), or polyvinylprrolidone (PVP, e.g. PVP-360 and PVP-40), or any combination thereof.

In some embodiments, the aggregation agent is configured to induce the aggregation of at least 50%, 60%, 70%, 80%, 90%, or 95% of the red blood cells in the sample within 1, 2, 5, 10, 20, 30, or 60 minutes, or in a time range between any of the two values.

FIG. 2 shows exemplary illustrations of the images of the sample. Panel (A) shows the sample before coagulation. Panel (B) shows the sample after coagulation. As indicated above, in some embodiments, during and/or after the deposition of the sample, the interference elements aggregate. Before aggregation, the interference elements are evenly distributed in the sample, making it difficult to analyze and/or measure the signals from the sample. In certain embodiments, as shown in panel (B), the interference elements aggregate and sample area can be divided into interference element rich regions and interference element poor regions. In certain embodiments, the detector, the imagers and the associated software thereof are configured to distinguish the signals from the interference element poor regions from the signals from the interference element rich region. In certain embodiments, the signals from the interference element poor region are specifically extracted, analyzed, and/or measure, providing a parameter that in some cases reflects the presence/amount of the analyte in the sample. In certain embodiments, the measurements from this approach include less bias compared with approaches that do not distinguish the interference element rich regions and interference element poor regions.

As depicted in the FIGS. 1 and 2, the interference element rich regions have significantly more interference elements than the interference element poor regions. In some embodiments, the interference element rich region has an area that is 30% or more, 40% or more, 50% or more, 60% or more, 70% or more, 80% or more, 90% or more, 95% or more, 99% or more, or 100% covered by the interference elements. In some embodiments, the interference element poor region has an area that is 30% or less, 20% or less, 15% or less, 10% or less, 5% or less, 2% or less, 1% or less, 0.1% or less, 0.01% or less, or 0% covered by the interference elements.

In some embodiments, the sample holder, when compressed, has a thickness of 500 nm or less, 1000 nm or less, 2 μm (micron) or less, 5 μm or less, 10 μm or less, 20 μm or less, 50 μm or less, 100 μm or less, 150 μm or less, 200 μm or less, 300 μm or less, 500 μm or less, 800 μm or less, 1 mm (millimeter) or less, 2 mm or less, 3 mm or less, 5 mm or less, 10 mm or less, or in a range between any two of these values.

FIG. 3 provides a schematic illustration showing some embodiments of the present invention, demonstrating the detector, the imager, and the sample holder that holds a sample, wherein the sample holder is a QMAX card (Q-card, Q: quantification; M: magnifying; A: adding reagents; X: acceleration; also known as compressed regulated open flow (CROF) device).

As shown in FIG. 3, the Q-card comprises a first plate and a second plate, and the two plates are relatively movable to each other into different configurations, including an open configuration and a closed configuration. The closed configuration is depicted in the figure, where the sample (not shown) is compressed by the two plates into a thin layer. In some embodiments, the thickness of the thin layer is 100 nm or less, 500 nm or less, 1 μm (micron) or less, 2 μm or less, 5 μm or less, 10 μm or less, 20 μm or less, 50 μm or less, 100 μm or less, 200 μm or less, 500 μm or less, 1 mm or less, 2 mm or less, 5 mm or less, 10 mm or less, or in a range between any two of these values. In preferred embodiments, the thickness of the thin layer is 1 μm or less, 2 μm or less, 5 μm or less, 10 μm or less, 20 μm or less, 50 μm or less, 100 μm or less, or in a range between any two of these values.

The embodiments in these applications herein incorporated can be regarded in combination with one another or as a single invention, rather than as discrete and independent filings. Moreover, the exemplary embodiments disclosed herein are applicable to embodiments including but not limited to: bio/chemical assays, QMAX cards and systems, QMAX with hinges, notches, recessed edges and sliders, assays and devices/apparatus with uniform sample thickness, smartphone detection systems, cloud computing designs, various detection methods, labels, capture agents and detection agents, analytes, diseases, applications, and samples; the various embodiments are disclosed, described, and/or referred to in the aforementioned applications, all of which are hereby incorporated in reference by their entireties.

In some embodiments, each of the plates has a thickness of 500 nm or less, 1000 nm or less, 2 μm (micron) or less, 5 μm or less, 10 μm or less, 20 μm or less, 50 μm or less, 100 μm or less, 150 μm or less, 200 μm or less, 300 μm or less, 500 μm or less, 800 μm or less, 1 mm (millimeter) or less, 2 mm or less, 3 mm or less, 5 mm or less, 10 mm or less, or in a range between any two of these values.

In some embodiments, each of the plates comprises a sample contact area, which is configured to contact the sample (but is not necessarily actually in contact with the sample in their entireties). In some embodiments, the area of the sample contact area is 1 mm² or less, 2 mm² or less, 5 mm² or less, 10 mm² or less, 20 mm² or less, 50 mm² or less, 100 mm² or less, 200 mm² or less, 500 mm² or less, 1000 mm² or less, 2000 mm² or less, 5000 mm² or less, or 10000 mm² or less, or in a range between any of the two values.

In some embodiments, the interference element rich region has an area that is 1 um² or less, 2 um² or less, 5 um² or less, 10 um² or less, 20 um² or less, 50 um² or less, 100 um² or less, 200 um² or less, 500 um² or less, 1 mm² or less, 2 mm² or less, 5 mm² or less, 10 mm² or less, 20 mm² or less, 50 mm² or less, 100 mm² or less, 200 mm² or less, 500 mm² or less, 1000 mm² or less, or in a range between any of the two values.

In some embodiments, the interference element poor region has an area that is 1 um² or less, 2 um² or less, 5 um² or less, 10 um² or less, 20 um² or less, 50 um² or less, 100 um² or less, 200 um² or less, 500 um² or less, 1 mm² or less, 2 mm² or less, 5 mm² or less, 10 mm² or less, 20 mm² or less, 50 mm² or less, 100 mm² or less, 200 mm² or less, 500 mm² or less, 1000 mm² or less, or in a range between any of the two values.

In some embodiments, the ratio of the area of the interference element rich region to the area of the interference element poor region is about 1/1000, 1/500, 1/200, 1/100, 1/50, 1/20, 1/10, 1/5, 1/4, 1/3, 1/2, 1, 2, 3, 4, 5, 10, 20, 50, 100, 200, 500, or 1000, or less than any of the values, or more than any of the values, or in a range between any of the values.

FIG. 4 shows an exemplary flow chart that demonstrates the process to conduct an assay that reduce the effects of interference elements. As shown in FIG. 4, in some embodiments, the method comprises:

i. obtaining a sample holder;

ii. depositing in the sample holder a sample that contains an analyte and one or more interference elements;

iii. imaging and identifying, with an imager and a software, (a) the regions in the sample that are occupied by the one or more has less inference elements concentration (“interference element rich region”) and/or (b) than another regions in the sample that are not occupied by the one or more inference elements (“interference element free poor region”), and/or (b) an interference element rich region; and

iv. measuring a signal related to the analyte in the interference element rich region and/or in the interference element free poor region.

In some embodiments, the signal related to analyte in the interference element poor region is measured. In some embodiments, the method further comprises calculating a concentration of the analyte in the sample based on the signal related to the analyte in the interference element rich region and/or in the interference element poor region. In some embodiments, the method further comprises calculating a concentration of the analyte in the sample based on the signal related to the analyte in the interference element poor region.

In some embodiments, the interference element poor region has an area that is less than 30%, 20%, 10%, 5%, 1%, or 0.1% covered by the interference element. In some embodiments, the sample is compressed by the sample holder into a layer of uniform thickness, and the method further comprises: calculating the volume of the sample based on an area of the sample layer. In some embodiments, the method further comprises: calculating the concentration of the analyte in the sample based on the signal related to the analyte in the interference element rich region and/or the interference element poor region, and the volume of the sample. In some embodiments, the method further comprises: calculating the concentration of the analyte in the sample based on the signal related to the analyte in the interference element poor region, and the volume of the sample in the interference element poor region.

In some embodiments, the detection and/or measurement of the analyte is based on the signals from the interference element rich region and the interference element poor region, the signals from the interference element rich region alone, or the interference element poor region alone. In certain embodiments, the detection and/or measurement of the analyte is based on the signals from the interference element poor region alone and the volume of the sample. In some embodiments, the detection and/or measurement of the analyte is based on the ratio of the interference element rich region to the interference element poor region.

In some embodiments, the sample area includes only the interference element rich region and the interference element poor region. In certain embodiments, the sample area does not include any other region besides the interference element rich region and the interference element poor region. In some embodiments, the sample area includes other regions beside the interference element rich region and the interference element poor region. For example, in certain embodiments, the sample area includes an exclusion area that is excluded from detection and/or measurement due to reasons such as but not limited poor and uneven illumination and presence of foreign (i.e. not part of sample) entity. In certain embodiments, the exclusion area can be viewed as another interference element rich region, especially when the exclusion is caused by the presence of certain entities (e.g. air bubble, etc.).

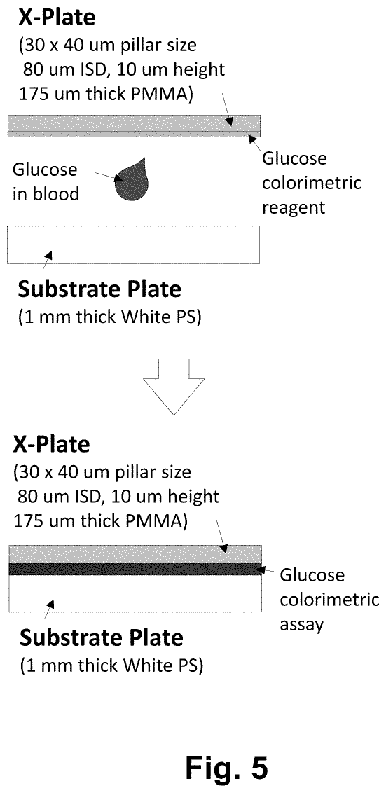

FIG. 5 shows an exemplary embodiment of the design of a QMAX card and the basic process to measure glucose levels in a blood sample. As shown in FIG. 5, the sample holder comprises a QMAX device, which includes a first plate (termed “X-plate”) and a second plate (termed “substrate plate”). The specific parameters for the first plate and the second plate are listed in FIG. 5, but variation can also apply in similar embodiments.

In some embodiments, as shown in FIG. 5, the assay is designed for the detection and/or measure of glucose in a blood sample. In some embodiments, the QMAX device also comprises reagents that are configured for the detection and/or measurement of glucose. In certain embodiments, the reagents are attached to one of the plates.

In some embodiments, blood sample that contains glucose can be deposited on one or both of the plates (e.g. the substrate plate). The plates are pressed against each other and the sample is compressed into a thin layer with uniform thickness. The thickness of the sample is regulated by the spacers that are fixed on one or both of the plates. The parameters of the spacers are listed in FIG. 5, but can vary based on the other conditions of the assay.

In certain embodiments, the reagents react with glucose and signals can be generated by the reaction. In certain embodiments, the reaction is a glucose oxidase-peroxide reaction that produce colored compounds that can be detected. In certain embodiments, the red blood cells in the blood sample causes difficulty in detecting and/or measuring the signal.

In some embodiments, aggregation reagents can be used to induce the aggregation of the red blood cells and/or coagulation of the blood, which is considered an interference element. In some embodiments, no aggregation reagent is used and the blood naturally coagulates. In some embodiments, the aggregation of the red blood cells creates interference element rich regions and interference element poor regions.

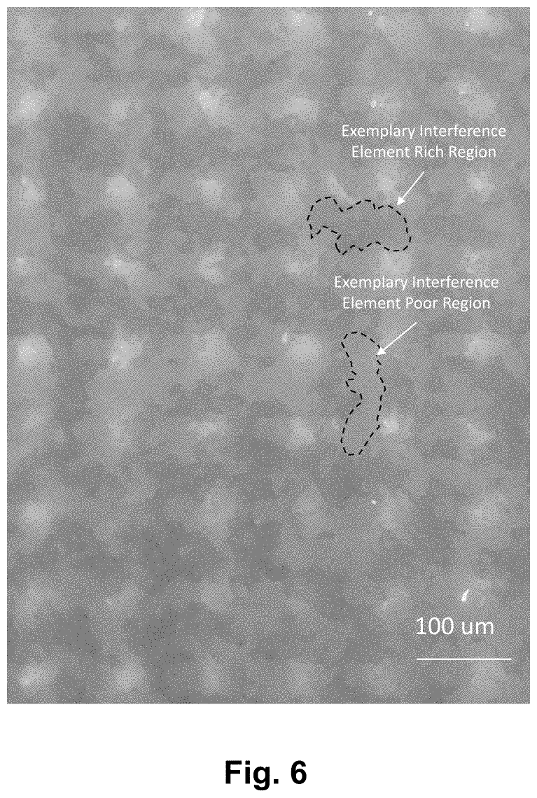

FIG. 6 shows an exemplary image of the sample, demonstrating the interference element rich region and the interference element poor region. As shown in FIG. 6, in some embodiments, the sample image includes interference element rich regions and interference poor regions. The regions can be identified by the imager and/or detector, with the associated software. In some embodiments, the regions can be defined according to raw signal intensity or the contrast of raw signal intensity between neighboring areas.

It should be noted that the exemplary interference element rich region and exemplary interference element poor region as shown in FIG. 5 are used for only for the demonstration of different regions. The specifics of the regions for analysis and/or measurement of the analyte can vary according to the specific parameters of the assay, such as but not limited to illumination intensity, sample thickness, reagent concentration etc. The example shown here does not in any way limit the method and/or device/apparatus that can be used to identify, delineate, and/or distinguish the interference element rich region and the interference element poor region.

### Computer Control Systems

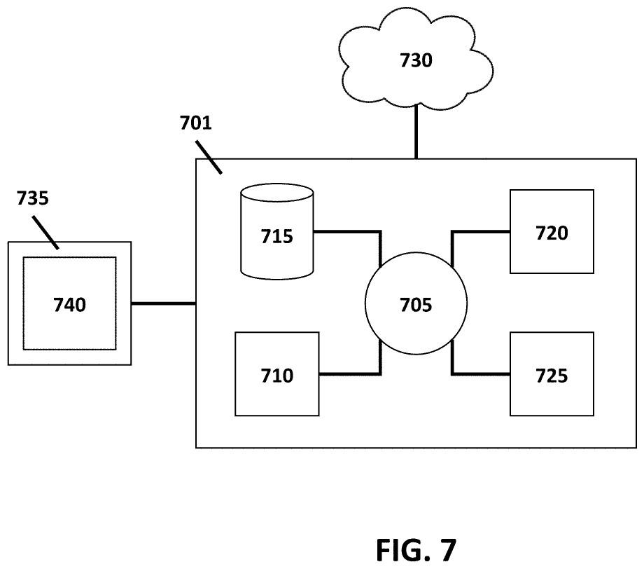

The present disclosure provides computer control systems that are programmed to implement methods of the disclosure. FIG. 7 shows a computer system 701 that is programmed or otherwise configured to analyze a sample. In some aspects, the computer system 701 can regulate various aspects of detecting the presence, absence and/or concentration of one or more analytes in a sample. For example, in some aspects, the computer system can be configured to identify (a) a region in the sample that has less interference element concentration (“interference element poor region”) than another region in the sample (“interference element rich region”), and/or (b) an interference element rich region, and/or (c) detect a signal related to the analyte in the interference element poor region and/or in the interference element rich region. The computer system 701 can be an electronic device of a user or a computer system that is remotely located with respect to the electronic device. The electronic device can be a mobile electronic device.

The computer system 701 includes a central processing unit (CPU, also “processor” and “computer processor” herein) 705, which can be a single core or multi core processor, or a plurality of processors for parallel processing. The computer system 701 also includes memory or memory location 710 (e.g., random-access memory, read-only memory, flash memory), electronic storage unit 715 (e.g., hard disk), communication interface 720 (e.g., network adapter) for communicating with one or more other systems, and peripheral devices 725, such as cache, other memory, data storage and/or electronic display adapters. The memory 710, storage unit 715, interface 720 and peripheral devices 725 are in communication with the CPU 705 through a communication bus (solid lines), such as a motherboard. The storage unit 715 can be a data storage unit (or data repository) for storing data. The computer system 701 can be operatively coupled to a computer network (“network”) 730 with the aid of the communication interface 720. The network 730 can be the Internet, an internet and/or extranet, or an intranet and/or extranet that is in communication with the Internet. The network 730 in some cases is a telecommunication and/or data network. The network 730 can include one or more computer servers, which can enable distributed computing, such as cloud computing. The network 730, in some cases with the aid of the computer system 701, can implement a peer-to-peer network, which may enable devices coupled to the computer system 701 to behave as a client or a server.

The CPU 705 can execute a sequence of machine-readable instructions, which can be embodied in a program or software. The instructions may be stored in a memory location, such as the memory 710. The instructions can be directed to the CPU 705, which can subsequently program or otherwise configure the CPU 705 to implement methods of the present disclosure. Examples of operations performed by the CPU 705 can include fetch, decode, execute, and writeback.

The CPU 705 can be part of a circuit, such as an integrated circuit. One or more other components of the system 701 can be included in the circuit. In some cases, the circuit is an application specific integrated circuit (ASIC).

The storage unit 715 can store files, such as drivers, libraries and saved programs. The storage unit 715 can store user data, e.g., user preferences and user programs. The computer system 701 in some cases can include one or more additional data storage units that are external to the computer system 701, such as located on a remote server that is in communication with the computer system 701 through an intranet or the Internet.

The computer system 701 can communicate with one or more remote computer systems through the network 730. For instance, the computer system 701 can communicate with a remote computer system of a user (e.g., a personal computer or a server comprising a training data set). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 701 via the network 730.

Methods as described herein can be implemented by way of machine (e.g., computer processor) executable code stored on an electronic storage location of the computer system 701, such as, for example, on the memory 710 or electronic storage unit 715. The machine executable or machine readable code can be provided in the form of software. During use, the code can be executed by the processor 705. In some cases, the code can be retrieved from the storage unit 715 and stored on the memory 710 for ready access by the processor 705. In some situations, the electronic storage unit 715 can be precluded, and machine-executable instructions are stored on memory 710.

### Machine Learning and Artificial Intelligence

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 701, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 701 can include or be in communication with an electronic display 735 that comprises a user interface (UI) 740 for providing, for example, a reading of the amount of one or more analytes present in a sample. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 705. The algorithm can, for example, employ artificial intelligence and/or machine learning and/or information of the spacer (e.g., height, width, or density) to detect the presence, absence or amount of one or more analytes in a sample.

In accordance with embodiments of the present disclosure, the algorithm can comprise a machine learning approach to detect one or more analytes in a sample (e.g., a whole blood sample). In general, any known machine learning approach can be utilized in practicing the present invention. For example, in one embodiment, non-negative matrix factorization can be utilized as a machine learning approach to decompose, or deconvolute, an observed matrix and identify underlying signatures prevalent in the dataset. To infer underlying signatures associated with the presence and/or absence of one or more analytes, we can decompose a matrix constructed of samples to explain the observed frequency contexts as a combination of the underlying signatures and the exposure each patient has to those signatures. In another embodiment, principal components analysis or vector quantization can be used.

In accordance with embodiments of the present disclosure, the algorithm can comprise an artificial intelligence and/or neural network approach to detect one or more analytes in a sample (e.g., a whole blood sample). Machine learning methods can be used to generate models that call the presence of an analyte in an image of a sample (taken by the imager) with higher accuracy than a heuristic method, and, optionally, providing a confidence level of the call. Such models can be generated by providing a machine learning unit with training data in which the expected output is known in advance, e.g. an output in which it is known that 99% of a given analyte have a specific diameter. Any metric may be used. For example, the shape (e.g., circular or non-circular), the size (e.g., greater than or less than 10 um), the color (e.g., red, orange, yellow, green, blue, or purple), and/or the light transmission properties of the analyte (e.g., opaque or non-opaque).

A training set can be provided as follows. A plurality of presumably homogenous normal samples comprising one or more analytes may be imaged. These samples can be, for example, whole blood from individuals who do not have a condition, e.g., influenza. This provides a set of images in which the number, size, shape, and/or transparency of blood cells (e.g., white blood cells) examined is expected to substantially uniform for healthy individuals. This can produce, for each sample, a vector indicating, the counts of the total number of cells, and/or the number of cells of a given size, shape or transparency against which a test sample can be compared.

### Examples of Present Invention

A1-1. An apparatus for assaying a sample that contains an analyte and interference elements, comprising:

a sample holder that is configured to hold a sample that contains an analyte and one or more interference elements;

an imager and a software that are configured to identify (a) a region in the sample that has less interference element concentration (“interference element poor region”) than another region in the sample (“interference element rich region”), and/or (b) an interference element rich region; and

a detector that is configured to detect a signal related to the analyte in the interference element poor region and/or in the interference element rich region.

A1-2. An apparatus for assaying a sample that contains an analyte and interference elements, comprising:

a sample holder that is configured to hold a sample that contains an analyte and one or more interference elements; and

an imager and a software that are configured to identify a region in the sample that has less interference element concentration (“interference element poor region”) than another region in the sample (“interference element rich region”); and

a detector that is configured to detect a signal related to the analyte in the interference element poor region.

A1-3. An apparatus for assaying a liquid sample that contains an analyte and interference elements, comprising:

a sample holder that comprises a first plate and a second plate and is configured to hold a sample that contains an analyte and one or more interference elements, wherein:
i. at least a part of the sample is between the first plate and second plate; and
ii. one or both of the plates are configured to allow the at least a part of the sample visible through the one or both of the plates;

an imager and a software that are configured to identify, in the at least a part of sample, a region that has less interference element concentration (“interference element poor region”) than another region in the sample layer (“interference element rich region”); and

a detector that is configured to detect a signal related to the analyte in an interference element poor region.

A1-4. An apparatus for assaying a liquid sample that contains an analyte and interference elements, comprising:

a sample holder that comprises a first plate, a second plate, and spacers and is configured to hold a sample that contains an analyte and one or more interference elements, wherein:
i. the first plate and second plate moveable relative to each other;
ii. the spacers are fixed on one or both of the plates and have a uniform height;
ii. the first plate and second plate are configured to compress the sample into a layer of uniform thickness that substantially equals the height of the spacers;

an imager and a software that are configured to identify a region in the sample layer that has less interference element concentration (“interference element poor region”) than another region in the sample layer (“interference element rich region”); and a detector that is configured to detect a signal related to the analyte in the interference element poor region.

A1-5. A kit for assaying a sample that contains an analyte and interference elements, comprising:

an apparatus of any prior embodiment; and

an aggregation reagent that causes or assists a sample to have a region that has less interference element concentration (“interference element poor region”) than another region in the sample (“interference element rich region”).

A2-1. A method for assaying a sample that contains an analyte and interference elements, comprising:
i. obtaining a sample holder;
ii. depositing in the sample holder a sample that contains an analyte and one or more interference elements;
iii. imaging and identifying, with an imager and a software, (a) the regions in the sample that has an inference element concentration (“interference element poor region”) substantially less than that in other region(s) (“interference element rich region”), and/or (b) an interference element rich region; and
iv. measuring a signal related to the analyte in the interference element rich region and/or in the interference element poor region.

A2-2. A method for assaying a sample that contains an analyte and interference elements, comprising:
i. obtaining a sample holder;
ii. depositing in the sample holder a sample that contains an analyte and one or more interference elements;
iii. imaging and identifying, with an imager and a software, (a) the regions in the sample that has less inference element concentration (“interference element rich region”) than anther region (“interference element poor region”); and
iv. measuring a signal related to the analyte in the interference element poor region.

A2-3. The method of any prior method embodiments, wherein it further comprises a step of adding an aggregation reagent that causes or assists a sample to have a region that has less interference element concentration (“interference element poor region”) than another region in the sample (“interference element rich region”).

A2-4. The method of any prior method embodiments, wherein the signal related to analyte in the interference element poor region is measured.

A2-5. The method of any prior method embodiments, further comprising calculating a concentration of the analyte in the sample based on the signal related to the analyte in the interference element rich region and/or in the interference element poor region.

A2-6. The method of any prior method embodiments, further comprising calculating a concentration of the analyte in the sample based on the signal related to the analyte in the interference element poor region.

A2-7. The method of any prior method embodiments, wherein the interference element poor region has an area that is less than 30%, 20%, 10%, 5%, 1%, or 0.1% covered by the interference element.

A2-8. The method of any prior method embodiments, wherein the sample is compressed by the sample holder into a layer of uniform thickness, and the method further comprises:

calculating the volume of the sample based on an area of the sample layer.

A2-9. The method of any prior method embodiments, further comprising: calculating the concentration of the analyte in the sample based on the signal related to the analyte in the interference element rich region and/or the interference element poor region, and the volume of the sample.

A2-10. The method of any prior method embodiments, further comprising: calculating the concentration of the analyte in the sample based on the signal related to the analyte in the interference element poor region, and the volume of the sample in the interference element poor region.

### General Elements

A3-1. The apparatus, kit, or method of any prior embodiments, wherein the detector is a part or a whole of the imager

A3-2. The apparatus, kit, or method of any prior embodiments, wherein the detector is a separate device from the imager

A3-3. The apparatus, kit, or method of any prior embodiments, wherein the apparatus further comprises an aggregation reagent that causes or assists a sample to has a region that has less interference element concentration (“interference element poor region”) than another region in the sample (“interference element rich region”).

A3-4. The apparatus, kit, or method of any prior embodiments, wherein the aggregation regent is coated on the sample holder.

A3-5. The apparatus, kit, or method of any prior embodiments, wherein the aggregation reagent is coated on the sample holder, and the aggregation reagent is a dry reagent.

A3-6. The apparatus, kit, or method of any prior embodiments, wherein the imager and the software are further configured to identify the interference element rich region.

A3-7. The apparatus, kit, or method of any prior embodiments, wherein the detector is further configured to detect a signal related to the analyte in the interference element rich region.

A3-8. The apparatus, kit, or method of any prior embodiments, wherein the detector is further configured to detect a signal related to the interference elements in the interference element rich region.

A3-9. The apparatus, kit, or method of any prior embodiments, wherein the sample holder is configured to compress the sample into a thin layer.

A3-10. The apparatus, kit, or method of any prior embodiments, wherein the sample holder is configured to compress the sample into a thin layer with uniform thickness.

A3-11. The apparatus, kit, or method of any prior embodiments, wherein the interference element rich region has an area that is at least 30%, 40%, 50%, 60%, 70%, 80%, 90%, or 99% covered by the interference elements.

A3-12. The apparatus, kit, or method of any prior embodiments, wherein the interference element poor region has an area that is less than 30%, 20%, 10%, 5%, 1%, or 0.1% covered by the interference elements.

A3-13. The apparatus, kit, or method of any prior embodiments, wherein the interference rich regions are formed without facilitation of factors not in the sample.

A3-14. The apparatus, kit, or method of any prior embodiments, wherein the interference rich regions are formed with facilitation of factors not in the sample.

### Microdomains

A4-1. The apparatus, kit, or method of any prior embodiments, wherein the interference element poor and/or rich regions in the sample form one or more microdomains, and wherein a microdomain is an interference element poor or region that has an average dimension of 800 um or less.

A4-2. The apparatus, kit, or method of any prior embodiments, wherein each of the one or more microdomain has an average dimension of less than 1 um, 10 um, 50 um, 100 um, 200 um, 250 um, 500 um, 600 um, 700 um, or 800 um, or in a range between any of the two values.

A4-3. The apparatus, kit, or method of any prior embodiments, wherein only the interference element poor regions in the sample form one or more microdomains, and wherein a microdomain is an interference element poor or region that has an average dimension of 800 um or less

A4-4. The apparatus, kit, or method of any prior embodiments, wherein only the interference element rich regions in the sample form one or more microdomains, and wherein a microdomain is an interference element poor or region that has an average dimension of 800 um or less.

A4-5. The apparatus, kit, or method of any prior embodiments, wherein each of the one or more microdomain has an average dimension of 700 um or less.

A4-6. The apparatus, kit, or method of any prior embodiments, wherein each of the one or more microdomain has an average dimension of 600 um or less.

A4-7. The apparatus, kit, or method of any prior embodiments, wherein each of the one or more microdomain has an average dimension of 500 um or less.

A4-8. The apparatus, kit, or method of any prior embodiments, wherein each of the one or more microdomain has an average dimension of 250 um or less.

A4-9. The apparatus, kit, or method of any prior embodiments, wherein each of the one or more microdomain has an average dimension of 100 um or less.

A4-10. The apparatus, kit, or method of any prior embodiments, wherein each of the one or more microdomain has an average dimension of 50 um or less.

A4-11. The apparatus, kit, or method of any prior embodiments, wherein each of the one or more microdomain has an average dimension of 10 um or less.

A4-12. The apparatus, kit, or method of any prior embodiments, wherein each of the one or more microdomain has an average dimension of 1 um or less.

A4-13. The apparatus, kit, or method of any prior embodiments, wherein each of the one or more microdomain has an average dimension in the range of 1-800 um, 50-800 um, 100-800 um, 250-800 um, 500-800 um, or 600-800 um.

A4-14. The apparatus, kit, or method of any prior embodiments, wherein each of the one or more microdomain has an average dimension in the range of 1-800 um, 1-700 um, 1-600 um, 1-500 um, 1-250 um, 1-100 um, 1-50 um, 1-25 um, or 1-10 um.

### Sample Thickness

A5-1. The apparatus, kit, or method of any prior embodiments, wherein at least part of the sample is compressed into a thin layer that has an average thickness of 500 um or less, 400 um or less, 300 um or less, 200 um or less, 175 um or less, 150 um or less, 125 um or less, 100 um or less, 75 um or less, 50 um or less, 40 um or less, 30 um or less, 20 um or less, 10 um or less, 5 um or less, 4 um or less, 3 um or less, 2 um or less, 1.8 um or less, 1.5 um or less, 1 um or less, 0.5 um or less, 0.2 um or less, 0.1 um or less, 50 nm or less, 20 nm or less, 10 nm or less, or in a range between any of the two values.

A5-2. The apparatus, kit, or method of any prior embodiments, wherein at least part of the sample is compressed into a thin layer, and wherein for a specific part of the sample that has an average thickness of 500 um or less, 400 um or less, 300 um or less, 200 um or less, 175 um or less, 150 um or less, 125 um or less, 100 um or less, 75 um or less, 50 um or less, 40 um or less, 30 um or less, 20 um or less, 10 um or less, 5 um or less, 4 um or less, 3 um or less, 2 um or less, 1.8 um or less, 1.5 um or less, 1 um or less, 0.5 um or less, 0.2 um or less, 0.1 um or less, 50 nm or less, 20 nm or less, 10 nm or less, or in a range between any of the two values, only the interference rich regions exist.

A5-3. The apparatus, kit, or method of any prior embodiments, wherein at least part of the sample is compressed into a thin layer, wherein for a specific part of the sample that has an average thickness of 500 um or less, 400 um or less, 300 um or less, 200 um or less, 175 um or less, 150 um or less, 125 um or less, 100 um or less, 75 um or less, 50 um or less, 40 um or less, 30 um or less, 20 um or less, 10 um or less, 5 um or less, 4 um or less, 3 um or less, 2 um or less, 1.8 um or less, 1.5 um or less, 1 um or less, 0.5 um or less, 0.2 um or less, 0.1 um or less, 50 nm or less, 20 nm or less, 10 nm or less, or in a range between any of the two values, only the interference poor regions exist.

A5-4. The apparatus, kit, or method of any prior embodiments, wherein at least part of the sample is compressed into a thin layer that has an average thickness of in a range of 0.5-2 um, 0.5-3 um, 0.5-5 um, 0.5-10 um, 0.5-20 um, 0.5-30 um, or 0.5-50 um.

A5-5. The apparatus, kit, or method of any prior embodiments, wherein at least part of the sample is compressed into a thin layer that has an average thickness of 500 um or less.

A5-6. The apparatus, kit, or method of any prior embodiments, wherein at least part of the sample is compressed into a thin layer that has an average thickness of 200 um or less.

A5-7. The apparatus, kit, or method of any prior embodiments, wherein at least part of the sample is compressed into a thin layer that has an average thickness of 100 um or less.

A5-8. The apparatus, kit, or method of any prior embodiments, wherein at least part of the sample is compressed into a thin layer that has an average thickness of 50 um or less.

A5-9. The apparatus, kit, or method of any prior embodiments, wherein at least part of the sample is compressed into a thin layer that has an average thickness of 25 um or less.

A5-10. The apparatus, kit, or method of any prior embodiments, wherein at least part of the sample is compressed into a thin layer that has an average thickness of 10 um or less.

A5-11. The apparatus, kit, or method of any prior embodiments, wherein at least part of the sample is compressed into a thin layer that has an average thickness of 5 um or less.

A5-12. The apparatus, kit, or method of any prior embodiments, wherein at least part of the sample is compressed into a thin layer that has an average thickness of 3 um or less.

A5-13. The apparatus, kit, or method of any prior embodiments, wherein at least part of the sample is compressed into a thin layer that has an average thickness of 2 um or less.

A5-14. The apparatus, kit, or method of any prior embodiments, wherein at least part of the sample is compressed into a thin layer that has an average thickness of 1 um or less.

A5-15. The apparatus, kit, or method of any prior embodiments, wherein at least part of the sample is compressed into a thin layer that has an average thickness of 500 nm or less.

A5-16. The apparatus, kit, or method of any prior embodiments, wherein at least part of the sample is compressed into a thin layer that has an average thickness of 100 nm or less.

A5-17. The apparatus, kit, or method of any prior embodiments, wherein at least part of the sample is compressed into a thin layer that has an average thickness in the range of 0.5-2 um, 0.5-3 um, or 0.5-5 um.

A5-18. The apparatus, kit, or method of any prior embodiments, wherein the average thickness of the layer of uniform thickness is in the range of 2 um to 2.2 um and the sample is blood.

A5-19. The apparatus, kit, or method of any prior embodiments, wherein the average thickness of the layer of uniform thickness is in the range of 2.2 um to 2.6 um and the sample is blood.

A5-20. The apparatus, kit, or method of any prior embodiments, wherein the average thickness of the layer of uniform thickness is in the range of 1.8 um to 2 um and the sample is blood.

A5-21. The apparatus, kit, or method of any prior embodiments, wherein the average thickness of the layer of uniform thickness is in the range of 2.6 um to 3.8 um and the sample is blood.

A5-22. The apparatus, kit, or method of any prior embodiments, wherein the average thickness of the layer of uniform thickness is in the range of 1.8 um to 3.8 um and the sample is whole blood without a dilution by another liquid.

A5-23. The apparatus, kit, or method of any prior embodiments, wherein the average thickness of the layer of uniform thickness is about equal to a minimum dimension of an analyte in the sample.

A5-24. The apparatus, kit, or method of any prior embodiments, wherein the final sample thickness device is configured to analyze the sample in 300 seconds or less.

A5-25. The apparatus, kit, or method of any prior embodiments, wherein the final sample thickness device is configured to analyze the sample in 180 seconds or less.

A5-26. The apparatus, kit, or method of any prior embodiments, wherein the final sample thickness device is configured to analyze the sample in 60 seconds or less.

A5-27. The apparatus, kit, or method of any prior embodiments, wherein the final sample thickness device is configured to analyze the sample in 30 seconds or less.

### Sample Types:

B1.1. The apparatus, kit, or method of any prior embodiments, wherein the sample is original, diluted, or processed forms of: bodily fluids, stool, amniotic fluid, aqueous humour, vitreous humour, blood, whole blood, fractionated blood, plasma, serum, breast milk, cerebrospinal fluid, cerumen, chyle, chime, endolymph, perilymph, feces, gastric acid, gastric juice, lymph, mucus, nasal drainage, phlegm, pericardial fluid, peritoneal fluid, pleural fluid, pus, rheum, saliva, sebum, semen, sputum, sweat, synovial fluid, tears, vomit, urine, or exhaled breath condensate.
B1.2. The apparatus, kit, or method of any prior embodiments, wherein the sample is original, diluted, or processed forms of blood.
B1.3. The apparatus, kit, or method of any prior embodiments, wherein the sample comprises whole blood.
B1.4. The apparatus, kit, or method of any prior embodiments, wherein the sample comprises an aggregation agent that induces aggregation of the interference elements.

### Analytes:

B2.1. The apparatus, kit, or method of any prior embodiments, wherein the analyte is a biomarker, an environmental marker, or a foodstuff marker.
B2.2. The apparatus, kit, or method of any prior embodiments, wherein the analyte is a biomarker indicative of the presence or severity of a disease or condition.
B2.3. The apparatus, kit, or method of any prior embodiments, wherein the analyte is a cell, a protein, or a nucleic acid.
B2.4. The apparatus, kit, or method of any prior embodiments, wherein the analyte comprises proteins, peptides, nucleic acids, synthetic compounds, inorganic compounds, organic compounds, bacteria, virus, cells, tissues, nanoparticles, and other molecules, compounds, mixtures and substances thereof.
B2.5. The apparatus, kit, or method of any prior embodiments, wherein the analyte is selected from Table B1, B2, B3 or B7 of PCT Application No. PCT/US2016/054,025.

### Interference Elements:

B3.1. The apparatus, kit, or method of any prior embodiments, wherein the interference elements generate signals that interfere with the signal from the analyte.
B3.2. The apparatus, kit, or method of any prior embodiments, wherein the interference elements comprise: cells, tissues, or chemical or biological molecules.
B3.3. The apparatus, kit, or method of any prior embodiments, wherein the sample comprises blood interference elements comprise blood cells.
B3.4. The apparatus, kit, or method of any prior embodiments, wherein the sample comprises blood interference elements comprise red blood cells.
B3.5. The apparatus, kit, or method of any prior embodiments, wherein the sample comprises whole blood interference elements comprise red blood cells.

### Sample Holder:

B4.1 The apparatus, kit, or method of any prior embodiments, wherein the sample holder comprises wells that configured to hold the sample.
B4.2 The apparatus, kit, or method of any prior embodiments, wherein the sample holder comprises a first plate, and a second plate, and spacers.
B4.3 The apparatus, kit, or method of any prior embodiments, wherein the sample holder comprises a first plate, a second plate, and spacers, wherein the spacers are configured to regulate a gap between the plates when the plates are pressed against each, compressing the sample into a thin layer.
B4.4 The apparatus, kit, or method of any prior embodiments, wherein the sample holder comprises a first plate, a second plate, and spacers, and wherein:

i. the plates are moveable relative to each other into different configurations, including an open configuration and a closed configuration;

ii. in the open configuration: the two plates are separated apart, the spacing between the plates is not regulated by the spacers, and the sample is deposited on one or both of the plates; and

iii. in the closed configuration, which is configured after the sample deposition in the open configuration: at least part of the sample is compressed by the two plates into a layer of highly uniform thickness and is substantially stagnant relative to the plates, wherein the uniform thickness of the layer is regulated by the plates and the spacers.

B4.5 The apparatus, kit, or method of any prior embodiments, wherein the sample holder comprises a Q-card, which comprises a first plate, a second plate, and spacers, wherein the spacers are configured to regulate a gap between the plates when the plates are pressed against each, compressing the sample into a thin layer.
B4.6 The apparatus, kit, or method of any prior embodiments, wherein

i. the sample holder comprises a first plate, a second plate, and spacers, wherein the spacers have a uniform height and a constant inter-spacer distance; and

ii. the sample is compressed by the sample holder into a thin layer with a uniform thickness that is regulated by the height of the spacers.

B4.7 The apparatus, kit, or method of any prior embodiments, wherein the sample is compressed into a layer of uniform thickness that substantially equals uniform height of spacers that are fixed to one or both of the plates.
B4.8 The apparatus, kit or method of any prior embodiments, wherein the sample is compressed into a layer of uniform thickness that has a variation of less than 15%, 10%, 5%, 2%, 1%, or in a range between any of the two values.
B4.9 The apparatus, kit, or method of any prior embodiments, wherein the sample, when compressed, has a thickness of 500 nm or less, 1000 nm or less, 2 um (micron) or less, 5 um or less, 10 um or less, 20 um or less, 50 um or less, 100 um or less, 150 um or less, 200 um or less, 300 um or less, 500 um or less, 800 um or less, 1 mm (millimeter) or less, 2 mm or less, 3 mm or less, 5 mm or less, 10 mm or less, or in a range between any two of these values.
B4.10 The apparatus, kit, or method of any prior embodiments, wherein the sample holder comprises a first plate and a second plate, wherein each of the plate has a thickness of 500 nm or less, 1000 nm or less, 2 um (micron) or less, 5 um or less, 10 um or less, 20 um or less, 50 um or less, 100 um or less, 150 um or less, 200 um or less, 300 um or less, 500 um or less, 800 um or less, 1 mm (millimeter) or less, 2 mm or less, 3 mm or less, 5 mm or less, 10 mm or less, or in a range between any two of these values.

### Aggregation Reagents

B5.1. The apparatus, kit, or method of any prior embodiments, wherein the aggregation agent induces aggregation of the interference elements.
B5.2 The apparatus, kit, or method of any prior embodiments, wherein the sample comprises blood and an aggregation agent that induces aggregation of red blood cells.
B5.3 The apparatus, kit, or method of any prior embodiments, wherein the aggregation agent comprises: fibrinogen (and subunits thereof), thrombin and prothrombin, certain dextran fractions (e.g. Dx-500, Dx-100, and Dx-70), poly(ethylene glycol), or polyvinylprrolidone (PVP, e.g. PVP-360 and PVP-40), or any combination thereof.
B5.4 The apparatus, kit, or method of any prior embodiments, wherein the aggregation agent is configured to induce the aggregation of at least 50%, 60%, 70%, 80%, 90%, or 95% of the red blood cells in the sample within 1, 2, 5, 10, 20, 30, or 60 minutes, or in a time range between any of the two values.

### Imager

B6.1 The apparatus, kit, or method of any prior embodiments, wherein the imager comprises a camera.
B6.2 The apparatus, kit, or method of any prior embodiments, wherein the imager is a part of the detector.
B6.3 The apparatus, kit, or method of any prior embodiments, wherein the imager is the entirety of the detector.
B6.4 The apparatus, kit, or method of any prior embodiments, wherein the imager is directed by the software to capture one or more images of the sample, identify the interference element regions and the interference element free regions, and digitally separate the interference element regions from the interference element free regions.
B6.5 The apparatus, kit, or method of any prior embodiments, wherein the imager comprises a filter that is configured to filter signals from the sample.
B6.6 The apparatus, kit, or method of any prior embodiments, wherein the imager comprises a light source that is configured to illuminate the sample.

### Detector:

B7.1 The apparatus, kit, or method of any prior embodiments, wherein the detector is a mobile device.
B7.3 The apparatus, kit, or method of any prior embodiments, wherein the detector is a smart phone.
B7.3 The apparatus, kit, or method of any prior embodiments, wherein the detector is a smart phone and the imager is a camera as part of the smart phone.
B7.4 The apparatus, kit, or method of any prior embodiments, wherein the detector comprises a display that is configured to show the presence and/or amount of the analyte.
B7.5 The apparatus, kit, or method of any prior embodiments, wherein the detector is configured to transmit detection results to a third party.

### Software

B8.1 The apparatus, kit, or method of any prior embodiments, wherein the software is stored in a storage unit, which is part of the detector.
B8.2 The apparatus, kit, or method of any prior embodiments, wherein the software is configured to direct the detector to display the presence and/or amount of the analyte.
B8.3 The apparatus, kit, or method of any prior embodiments, wherein the software is configured to direct the imager to calculate the combined signal of the analyte from the interference element free regions.
B8.4 The apparatus, kit, or method of any prior embodiments, wherein the software is configured to direct the imager to disregard the signal of the analyte from the interference element regions.
B8.5 The apparatus, kit, or method of any prior embodiments, wherein the software is configured to direct the imager to increase signal contrast of the signals from the interference element regions to the signals from the interference element free regions
B8.6 The apparatus, kit, or method of any prior embodiments, wherein the software is configured to direct the detector to calculate a ratio of the signal from the interference element regions to the interference element free regions.

### Fields and Applications:

B9.1 The apparatus, kit, or method of any prior embodiments, wherein the apparatus or method are used for detection of proteins, peptides, nucleic acids, synthetic compounds, inorganic compounds, organic compounds, bacteria, virus, cells, tissues, nanoparticles, and other molecules, compounds, mixtures and substances thereof.
B9.2 The apparatus, kit, or method of any prior embodiments, wherein the apparatus or method are used for diagnostics, management, and/or prevention of human diseases and conditions.
B9.3 The apparatus, kit, or method of any prior embodiments, wherein the apparatus or method are used for diagnostics, management, and/or prevention of veterinary diseases and conditions, or for diagnostics, management, and/or prevention of plant diseases and conditions.
B9.4 The apparatus, kit, or method of any prior embodiments, wherein the apparatus or method are used for environments testing and decontamination.
B9.5 The apparatus, kit, or method of any prior embodiments, wherein the apparatus or method are used for agricultural or veterinary applications.
B9.6 The apparatus, kit, or method of any prior embodiments, wherein the apparatus or method are used for food testing.
B9.7 The apparatus, kit, or method of any prior embodiments, wherein the apparatus or method are used for drug testing and prevention.
B9.8 The apparatus, kit, or method of any prior embodiments, wherein the apparatus or method are used for detecting and/or measuring an analyte in blood.
B9.9 The apparatus, kit, or method of any prior embodiments, wherein the apparatus or method are used for a colorimetric assay.
B9.10 The apparatus, kit, or method of any prior embodiments, wherein the apparatus or method are used for a fluorescence assay.

### Signal Related to Analyte

B10.1 The apparatus, kit, or method of any prior embodiments, wherein the signal related to the analyte is an electrical signal or an optical signal.
B10.2 The apparatus, kit, or method of any prior embodiments, wherein the signal related to the analyte is an optical signal that allows the imager to capture images of the interference element rich region and the interference element poor region.
B10.3 The apparatus, kit, or method of any prior embodiments, wherein the signal related to the analyte is from a colorimetric reaction.
B10.4 The apparatus, kit, or method of any prior embodiments, wherein the signal related to the analyte is produced by illuminating the sample with an illumination source.

### Spacers and Plates

B11.1 The apparatus, kit, or method of any prior embodiments, wherein the plates are movable relative to each.
B11.1 The apparatus, kit, or method of any prior embodiments, wherein the spacers are fixed on one or both of the plates and have a uniform height.
B11.1 The apparatus, kit, or method of any prior embodiments, wherein the first plate and second plate are configured to compress the sample into a layer of uniform thickness that substantially equals the height of the spacers.
B11.1 The apparatus, kit, or method of any prior embodiments, wherein the spacers have a uniform height of 1 mm or less, 500 um or less, 400 um or less, 300 um or less, 200 um or less, 175 um or less, 150 um or less, 125 um or less, 100 um or less, 75 um or less, 50 um or less, 40 um or less, 30 um or less, 20 um or less, 10 um or less, 5 um or less, 4 um or less, 3 um or less, 2 um or less, 1.8 um or less, 1.5 um or less, 1 um or less, 0.5 um or less, 0.2 um or less, 0.1 um or less, 50 nm or less, 20 nm or less, 10 nm or less, or in a range between any of the two values.
B11.2 The apparatus, kit, or method of any prior embodiments, wherein the spacers have a uniform height in the range of 0.5-2 um, 0.5-3 um, 0.5-5 um, 0.5-10 um, 0.5-20 um, 0.5-30 um, or 0.5-50 um.
B11.3 The apparatus, kit, or method of any prior embodiments, wherein at least one of the plates has a thickness of 100 mm or less, 50 mm or less, 25 mm or less, 10 mm or less, 5 mm or less, 1 mm or less, 500 um or less, 400 um or less, 300 um or less, 200 um or less, 175 um or less, 150 um or less, 125 um or less, 100 um or less, 75 um or less, 50 um or less, 40 um or less, 30 um or less, 20 um or less, 10 um or less, 5 um or less, 4 um or less, 3 um or less, 2 um or less, 1.8 um or less, 1.5 um or less, 1 um or less, 0.5 um or less, 0.2 um or less, or 0.1 um or less, or in a range between any of the two values.
B11.4 The apparatus, kit, or method of any prior embodiments, wherein at least one of the plates has a thickness in the range of 0.5 to 1.5 mm; around 1 mm; in the range of 0.15 to 0.2 mm; or around 0.175 mm.
B11.5 The apparatus, kit, or method of any prior embodiments, wherein at least one of the plates has a lateral area of 1 mm² or less, 10 mm² or less, 25 mm² or less, 50 mm² or less, 75 mm² or less, 1 cm² (square centimeter) or less, 2 cm² or less, 3 cm² or less, 4 cm² or less, 5 cm² or less, 10 cm² or less, 100 cm² or less, 500 cm² or less, 1000 cm² or less, 5000 cm² or less, 10,000 cm² or less, 10,000 cm² or less, or in a range between any two of these values
B11.6 The apparatus, kit, or method of any prior embodiments, wherein at least one of the plates has a lateral area of in the range of 500 to 1000 mm²; or around 750 mm²
B11.7 The apparatus, kit, or method of any prior embodiments, wherein the Young's modulus of the spacers times the filling factor of the spacers is equal or larger than 10 MPa, wherein the filling factor is the ratio of the spacer area in contact with the layer of uniform thickness to the total plate area in contact with the layer of uniform thickness.
B11.8 The apparatus, kit, or method of any prior embodiments, wherein the thickness of the flexible plate times the Young's modulus of the flexible plate is in the range 60 to 750 GPa-um.
B11.9 The apparatus, kit, or method of any prior embodiments, wherein for a flexible plate, the fourth power of the inter-spacer-distance (ISD) divided by the thickness of the flexible plate (h) and the Young's modulus (E) of the flexible plate, ISD⁴/(hE), is equal to or less than 10⁶ um³/GPa.
B11.10 The apparatus, kit, or method of any prior embodiments, wherein one or both plates comprises a location marker, either on a surface of or inside the plate, that provide information of a location of the plate.
B11.11 The apparatus, kit, or method of any prior embodiments, wherein one or both plates comprises a scale marker, either on a surface of or inside the plate, that provide information of a lateral dimension of a structure of the sample and/or the plate.
B11.12 The apparatus, kit, or method of any prior embodiments, wherein one or both plates comprises an image marker, either on a surface of or inside the plate, that assists an imaging of the sample.
B11.13 The apparatus, kit, or method of any prior embodiments, wherein the inter-spacer distance is in the range of 7 um to 50 um.
B11.14 The apparatus, kit, or method of any prior embodiments, wherein the inter-spacer distance is in the range of 50 um to 120 um.
B11.15 The apparatus, kit, or method of any prior embodiments, wherein the inter-spacer distance is in the range of 120 um to 200 um.
B11.16 The apparatus, kit, or method of any prior embodiments, wherein the spacers are pillars with a cross-sectional shape selected from round, polygonal, circular, square, rectangular, oval, elliptical, or any combination of the same.
B11.17 The apparatus, kit, or method of any prior embodiments, wherein the spacers have a pillar shape and have a substantially flat top surface, wherein, for each spacer, the ratio of the lateral dimension of the spacer to its height is at least 1.
B11.18 The apparatus, kit, or method of any prior embodiments, wherein each spacer has the ratio of the lateral dimension of the spacer to its height is at least 1.
B11.19 The apparatus, kit, or method of any prior embodiments, wherein the minimum lateral dimension of spacer is less than or substantially equal to the minimum dimension of an analyte in the sample.
B11.20 The apparatus, kit, or method of any prior embodiments, wherein the minimum lateral dimension of spacer is in the range of 0.5 um to 100 um.
B11.21 The apparatus, kit, or method of any prior embodiments, wherein the minimum lateral dimension of spacer is in the range of 0.5 um to 10 um.
B11.22 The apparatus, kit, or method of any prior embodiments, wherein the spacers have a pillar shape, and the sidewall corners of the spacers have a round shape with a radius of curverture at least 1 □m.
B11.23 The apparatus, kit, or method of any prior embodiments, wherein the spacers have a density of at least 100/mm².
B11.24 The apparatus, kit, or method of any prior embodiments, wherein the spacers have a density of at least 1000/mm².
B11.25 The apparatus, kit, or method of any prior embodiments, wherein at least one of the plates is transparent
B11.26 The apparatus, kit, or method of any prior embodiments, wherein at least one of the plates is made from a flexible polymer.
B11.27 The apparatus, kit, or method of any prior embodiments, wherein, for a pressure that compresses the plates, the spacers are not compressible and/or, independently, only one of the plates is flexible.
B11.28 The apparatus, kit, or method of any prior embodiments, wherein the flexible plate has a thickness in the range of 10 um to 200 um.
B11.29 The apparatus, kit, or method of any prior embodiments, wherein the variation of sample thickness is less than 30%.
B11.30 The apparatus, kit, or method of any prior embodiments, wherein the variation of sample thickness is less than 10%.
B11.31 The apparatus, kit, or method of any prior embodiments, wherein the variation of sample thickness is less than 5%.
B11.32 The apparatus, kit, or method of any prior embodiments, wherein the first and second plates are connected and are configured to be changed from the open configuration to the closed configuration by folding the plates.
B11.33 The apparatus, kit, or method of any prior embodiments, wherein the first and second plates are connected by a hinge and are configured to be changed from the open configuration to the closed configuration by folding the plates along the hinge.
B11.34 The apparatus, kit, or method of any prior embodiments, wherein the first and second plates are connected by a hinge that is a separate material to the plates, and are configured to be changed from the open configuration to the closed configuration by folding the plates along the hinge.
B11.35 The apparatus, kit, or method of any prior embodiments, wherein the first and second plates are made in a single piece of material and are configured to be changed from the open configuration to the closed configuration by folding the plates.
B11.36 The apparatus, kit, or method of any prior embodiments, wherein the layer of uniform thickness sample is uniform over a lateral area that is at least 1 mm².
B11.37 The apparatus, kit, or method of any prior embodiments, wherein the spacers are fixed on a plate by directly embossing the plate or injection molding of the plate.
B11.38 The apparatus, kit, or method of any prior embodiments, wherein the materials of the plate and the spacers are selected from polystyrene, PMMA, PC, COC, COP, or another plastic.
C1.1 A device or system comprising a non-transitory, computer readable medium comprising machine-executable code that, upon execution by a computer processor, implements any method of the present disclosure.
C1.2 The device or system of any prior embodiment, wherein the machine-executable code comprises machine learning.
C1.3 The device or system of can prior embodiment, wherein the machine-executable code comprises artificial intelligence.
C1.4 The device or system of any prior embodiment, wherein the machine-executable code comprises an algorithm for using a spacer height, width, and/or density to determine the presence, absence or concentration of one or more analytes in a sample.
C2.1 A non-transitory computer-readable medium comprising machine executable code that, upon execution by one or more computer processors, implements a method for detecting one or more analytes in a sample, the method comprising:
i. generating training data;
ii. in computer memory, generating a machine learning unit comprising one or more output calls for each of the one or more analytes in a sample, the sample comprising the one or more analytes and one or more interference elements, the sample at least partially contained within a sample holder that comprises a first plate and a second plate, wherein at least a part of the sample is between the first plate and second plate, and wherein one or both of the plates are configured to allow the at least a part of the sample visible through the one or both of the plates;
iii. training the machine learning unit with a training set of samples, wherein the trained machine learning unit is configured to detect the one or more analytes from the sample of a subject using an imager and a detector,
wherein the sample comprises a mixture of analytes,
wherein the imager is configured to identify, in the at least a part of sample, a region that has less interference element concentration (“interference element poor region”) than another region in the sample layer (“interference element rich region”), and
wherein the detector is configured to detect a signal related to the analyte in an interference element poor region.
   C3.1 A method for detecting one or more analytes in a sample, the method comprising:
i. generating training data;
ii. in computer memory, generating a machine learning unit comprising one or more output calls for each of the one or more analytes in a sample, the sample comprising the one or more analytes and one or more interference elements, the sample at least partially contained within a sample holder that comprises a first plate and a second plate, wherein at least a part of the sample is between the first plate and second plate, and wherein one or both of the plates are configured to allow the at least a part of the sample visible through the one or both of the plates;
iii. training the machine learning unit with a training set of samples, wherein the trained machine learning unit is configured to detect the one or more analytes from the sample of a subject using an imager and a detector,
wherein the sample comprises a mixture of analytes,
wherein the imager is configured to identify, in the at least a part of sample, a region that has less interference element concentration (“interference element poor region”) than another region in the sample layer (“interference element rich region”), and
wherein the detector is configured to detect a signal related to the analyte in an interference element poor region.
   C4.1 A system for detecting one or more analytes in a sample, the system comprising:
i. computer memory for containing a machine learning unit to detect the one or more analytes in the sample, the sample comprising the one or more analytes and one or more interference elements, the sample at least partially contained within a sample holder that comprises a first plate and a second plate, wherein at least a part of the sample is between the first plate and second plate, and wherein one or both of the plates are configured to allow the at least a part of the sample visible through the one or both of the plates;
ii. one or more computer processors that are individually or collectively programmed to:
   a. generate training data;
   b. generate a machine learning unit comprising one or more output calls for each of the one or more analytes in a sample;
   c. train the machine learning unit with a training set of samples; and
   d. apply the machine learning unit to detect the one or more analytes from the sample of a subject, wherein the sample comprises a mixture of analytes;
iii. an imager configured to identify, in the at least a part of sample, a region that has less interference element concentration (“interference element poor region”) than another region in the sample layer (“interference element rich region”); and
iv. a detector configured to detect a signal related to the analyte in an interference element poor region.

### Related Documents and Additional Examples

The present invention includes a variety of embodiments, which can be combined in multiple ways as long as the various components do not contradict one another. The embodiments should be regarded as a single invention file: each filing has other filing as the references and is also referenced in its entirety and for all purpose, rather than as a discrete independent. These embodiments include not only the disclosures in the current file, but also the documents that are herein referenced, incorporated, or to which priority is claimed.

### (1) Definitions

The terms used in describing the devices/apparatus, systems, and methods herein disclosed are defined in the current application, or in PCT Application (designating U.S.) Nos. PCT/US2016/045437 and PCT/US0216/051775, which were respectively filed on Aug. 10, 2016 and Sep. 14, 2016, U.S. Provisional Application No. 62/456,065, which was filed on Feb. 7, 2017, U.S. Provisional Application No. 62/456,287, which was filed on Feb. 8, 2017, and U.S. Provisional Application No. 62/456,504, which was filed on Feb. 8, 2017, all of which applications are incorporated herein in their entireties for all purposes.

The terms “CROF Card (or card)”, “COF Card”, “QMAX-Card”, “Q-Card”, “CROF device”, “COF device”, “QMAX-device”, “CROF plates”, “COF plates”, and “QMAX-plates” are interchangeable, except that in some embodiments, the COF card does not comprise spacers; and the terms refer to a device that comprises a first plate and a second plate that are movable relative to each other into different configurations (including an open configuration and a closed configuration), and that comprises spacers (except some embodiments of the COF card) that regulate the spacing between the plates. The term “X-plate” refers to one of the two plates in a CROF card, wherein the spacers are fixed to this plate. More descriptions of the COF Card, CROF Card, and X-plate are given in the provisional application Ser. No. 62/456,065, filed on Feb. 7, 2017, which is incorporated herein in its entirety for all purposes.

### (2) Sample

The devices/apparatus, systems, and methods herein disclosed can be applied to manipulation and detection of various types of samples. The samples are herein disclosed, listed, described, and/or summarized in PCT Application (designating U.S.) Nos. PCT/US2016/045437 and PCT/US0216/051775, which were respectively filed on Aug. 10, 2016 and Sep. 14, 2016, U.S. Provisional Application No. 62/456,065, which was filed on Feb. 7, 2017, U.S. Provisional Application No. 62/456,287, which was filed on Feb. 8, 2017, and U.S. Provisional Application No. 62/456,504, which was filed on Feb. 8, 2017, all of which applications are incorporated herein in their entireties for all purposes.

The devices, apparatus, systems, and methods herein disclosed can be used for samples such as but not limited to diagnostic samples, clinical samples, environmental samples and foodstuff samples. The types of sample include but are not limited to the samples listed, described and/or summarized in PCT Application (designating U.S.) Nos. PCT/US2016/045437 and PCT/US0216/051775, which were respectively filed on Aug. 10, 2016 and Sep. 14, 2016, and are hereby incorporated by reference by their entireties.

For example, in some embodiments, the devices, apparatus, systems, and methods herein disclosed are used for a sample that includes cells, tissues, bodily fluids and/or a mixture thereof. In some embodiments, the sample comprises a human body fluid. In some embodiments, the sample comprises at least one of cells, tissues, bodily fluids, stool, amniotic fluid, aqueous humour, vitreous humour, blood, whole blood, fractionated blood, plasma, serum, breast milk, cerebrospinal fluid, cerumen, chyle, chime, endolymph, perilymph, feces, gastric acid, gastric juice, lymph, mucus, nasal drainage, phlegm, pericardial fluid, peritoneal fluid, pleural fluid, pus, rheum, saliva, sebum, semen, sputum, sweat, synovial fluid, tears, vomit, urine, and exhaled breath condensate.

In some embodiments, the devices, apparatus, systems, and methods herein disclosed are used for an environmental sample that is obtained from any suitable source, such as but not limited to: river, lake, pond, ocean, glaciers, icebergs, rain, snow, sewage, reservoirs, tap water, drinking water, etc.; solid samples from soil, compost, sand, rocks, concrete, wood, brick, sewage, etc.; and gaseous samples from the air, underwater heat vents, industrial exhaust, vehicular exhaust, etc. In certain embodiments, the environmental sample is fresh from the source; in certain embodiments, the environmental sample is processed. For example, samples that are not in liquid form are converted to liquid form before the subject devices, apparatus, systems, and methods are applied.

In some embodiments, the devices, apparatus, systems, and methods herein disclosed are used for a foodstuff sample, which is suitable or has the potential to become suitable for animal consumption, e.g., human consumption. In some embodiments, a foodstuff sample includes raw ingredients, cooked or processed food, plant and animal sources of food, preprocessed food as well as partially or fully processed food, etc. In certain embodiments, samples that are not in liquid form are converted to liquid form before the subject devices, apparatus, systems, and methods are applied.

The subject devices, apparatus, systems, and methods can be used to analyze any volume of the sample. Examples of the volumes include, but are not limited to, about 10 mL or less, 5 mL or less, 3 mL or less, 1 microliter (μL, also “uL” herein) or less, 500 μL, or less, 300 μL or less, 250 μL, or less, 200 μL, or less, 170 μL, or less, 150 μL, or less, 125 μL or less, 100 μL or less, 75 μL or less, 50 μL, or less, 25 μL or less, 20 μL, or less, 15 μL or less, 10 μL or less, 5 μL or less, 3 μL or less, 1 μL or less, 0.5 μL or less, 0.1 μL or less, 0.05 μL or less, 0.001 μL or less, 0.0005 μL or less, 0.0001 μL or less, 10 pL or less, 1 pL or less, or a range between any two of the values.

In some embodiments, the volume of the sample includes, but is not limited to, about 100 μL or less, 75 μL or less, 50 μL, or less, 25 μL, or less, 20 μL, or less, 15 μL, or less, 10 μL or less, 5 μL or less, 3 μL or less, 1 μL or less, 0.5 μL, or less, 0.1 μL, or less, 0.05 μL, or less, 0.001 μL or less, 0.0005 μL, or less, 0.0001 μL or less, 10 pL or less, 1 pL or less, or a range between any two of the values. In some embodiments, the volume of the sample includes, but is not limited to, about 10 μL or less, 5 μL or less, 3 μL or less, 1 μL or less, 0.5 μL or less, 0.1 μL or less, 0.05 μL or less, 0.001 μL or less, 0.0005 μL or less, 0.0001 μL or less, 10 pL or less, 1 pL or less, or a range between any two of the values.

In some embodiments, the amount of the sample is about a drop of liquid. In certain embodiments, the amount of sample is the amount collected from a pricked finger or fingerstick. In certain embodiments, the amount of sample is the amount collected from a microneedle, micropipette or a venous draw.

In certain embodiments, the sample holder is configured to hold a fluidic sample. In certain embodiments, the sample holder is configured to compress at least part of the fluidic sample into a thin layer. In certain embodiments, the sample holder comprises structures that are configured to heat and/or cool the sample. In certain embodiments, the heating source provides electromagnetic waves that can be absorbed by certain structures in the sample holder to change the temperature of the sample. In certain embodiments, the signal sensor is configured to detect and/or measure a signal from the sample. In certain embodiments, the signal sensor is configured to detect and/or measure an analyte in the sample. In certain embodiments, the heat sink is configured to absorb heat from the sample holder and/or the heating source. In certain embodiments, the heat sink comprises a chamber that at least partly enclose the sample holder.

### (3) Q-Card, Spacers and Uniform Sample Thickness

The devices/apparatus, systems, and methods herein disclosed can include or use Q-cards, spacers, and uniform sample thickness embodiments for sample detection, analysis, and quantification. In some embodiments, the Q-card comprises spacers, which help to render at least part of the sample into a layer of high uniformity. The structure, material, function, variation and dimension of the spacers, as well as the uniformity of the spacers and the sample layer, are herein disclosed, listed, described, and/or summarized in PCT Application (designating U.S.) Nos. PCT/US2016/045437 and PCT/US0216/051775, which were respectively filed on Aug. 10, 2016 and Sep. 14, 2016, U.S. Provisional Application No. 62/456,065, which was filed on Feb. 7, 2017, U.S. Provisional Application No. 62/456,287, which was filed on Feb. 8, 2017, and U.S. Provisional Application No. 62/456,504, which was filed on Feb. 8, 2017, all of which applications are incorporated herein in their entireties for all purposes.

The term “open configuration” of the two plates in a QMAX process means a configuration in which the two plates are either partially or completely separated apart and the spacing between the plates is not regulated by the spacers

The term “closed configuration” of the two plates in a QMAX process means a configuration in which the plates are facing each other, the spacers and a relevant volume of the sample are between the plates, the relevant spacing between the plates, and thus the thickness of the relevant volume of the sample, is regulated by the plates and the spacers, wherein the relevant volume is at least a portion of an entire volume of the sample.

The term “a sample thickness is regulated by the plate and the spacers” in a QMAX process means that for a give condition of the plates, the sample, the spacer, and the plate compressing method, the thickness of at least a port of the sample at the closed configuration of the plates can be predetermined from the properties of the spacers and the plate.

The term “inner surface” or “sample surface” of a plate in a QMAX card refers to the surface of the plate that touches the sample, while the other surface (that does not touch the sample) of the plate is termed “outer surface”.

The term “height” or “thickness” of an object in a QMAX process refers to, unless specifically stated, the dimension of the object that is in the direction normal to a surface of the plate. For example, spacer height is the dimension of the spacer in the direction normal to a surface of the plate, and the spacer height and the spacer thickness means the same thing.

The term “area” of an object in a QMAX process refers to, unless specifically stated, the area of the object that is parallel to a surface of the plate. For example, spacer area is the area of the spacer that is parallel to a surface of the plate.

The term of QMAX card refers the device that perform a QMAX (e.g. CROF) process on a sample, and have or not have a hinge that connect the two plates.

The term “QMAX card with a hinge and “QMAX card” are interchangeable.

The term “angle self-maintain”, “angle self-maintaining”, or “rotation angle self-maintaining” refers to the property of the hinge, which substantially maintains an angle between the two plates, after an external force that moves the plates from an initial angle into the angle is removed from the plates.

In using QMAX card, the two plates need to be open first for sample deposition. However, in some embodiments, the QMAX card from a package has the two plates are in contact each other (e.g. a close position), and to separate them is challenges, since one or both plates are very thing. To facilitate an opening of the QMAX card, opening notch or notches are created at the edges or corners of the first plate or both places, and, at the close position of the plates, a part of the second plate placed over the opening notch, hence in the notch of the first plate, the second plate can be lifted open without a blocking of the first plate.

In the QMAX assay platform, a QMAX card uses two plates to manipulate the shape of a sample into a thin layer (e.g. by compressing). In certain embodiments, the plate manipulation needs to change the relative position (termed: plate configuration) of the two plates several times by human hands or other external forces. There is a need to design the QMAX card to make the hand operation easy and fast.

In QMAX assays, one of the plate configurations is an open configuration, wherein the two plates are completely or partially separated (the spacing between the plates is not controlled by spacers) and a sample can be deposited. Another configuration is a closed configuration, wherein at least part of the sample deposited in the open configuration is compressed by the two plates into a layer of highly uniform thickness, the uniform thickness of the layer is confined by the inner surfaces of the plates and is regulated by the plates and the spacers. In some embodiments, the average spacing between the two plates is more than 300 um.

In a QMAX assay operation, an operator needs to first make the two plates to be in an open configuration ready for sample deposition, then deposit a sample on one or both of the plates, and finally close the plates into a close position. In certain embodiments, the two plates of a QMAX card are initially on top of each other and need to be separated to get into an open configuration for sample deposition. When one of the plate is a thin plastic film (175 um thick PMA), such separation can be difficult to perform by hand. The present invention intends to provide the devices and methods that make the operation of certain assays, such as the QMAX card assay, easy and fast.

In some embodiments, the QMAX device comprises a hinge that connect two or more plates together, so that the plates can open and close in a similar fashion as a book. In some embodiments, the material of the hinge is such that the hinge can self-maintain the angle between the plates after adjustment. In some embodiments, the hinge is configured to maintain the QMAX card in the closed configuration, such that the entire QMAX card can be slide in and slide out a card slot without causing accidental separation of the two plates. In some embodiments, the QMAX device comprises one or more hinges that can control the rotation of more than two plates.

In some embodiments, the hinge is made from a metallic material that is selected from a group consisting of gold, silver, copper, aluminum, iron, tin, platinum, nickel, cobalt, alloys, or any combination of thereof. In some embodiments, the hinge comprises a single layer, which is made from a polymer material, such as but not limited to plastics. The polymer material is selected from the group consisting of acrylate polymers, vinyl polymers, olefin polymers, cellulosic polymers, noncellulosic polymers, polyester polymers, Nylon, cyclic olefin copolymer (COC), poly(methyl methacrylate) (PMMB), polycarbonate (PC), cyclic olefin polymer (COP), liquid crystalline polymer (LCP), polyamide (PB), polyethylene (PE), polyimide (PI), polypropylene (PP), poly(phenylene ether) (PPE), polystyrene (PS), polyoxymethylene (POM), polyether ether ketone (PEEK), polyether sulfone (PES), poly(ethylene phthalate) (PET), polytetrafluoroethylene (PTFE), polyvinyl chloride (PVC), polyvinylidene fluoride (PVDF), polybutylene terephthalate (PBT), fluorinated ethylene propylene (FEP), perfluoroalkoxyalkane (PFB), polydimethylsiloxane (PDMS), rubbers, or any combinations of thereof. In some embodiments, the polymer material is selected from polystyrene, PMMB, PC, COC, COP, other plastic, or any combination of thereof.

In essence, the term “spacers” or “stoppers” refers to, unless stated otherwise, the mechanical objects that set, when being placed between two plates, a limit on the minimum spacing between the two plates that can be reached when compressing the two plates together. Namely, in the compressing, the spacers will stop the relative movement of the two plates to prevent the plate spacing becoming less than a preset (i.e. predetermined) value.

The term “a spacer has a predetermined height” and “spacers have a predetermined inter-spacer distance” means, respectively, that the value of the spacer height and the inter spacer distance is known prior to a QMAX process. It is not predetermined, if the value of the spacer height and the inter-spacer distance is not known prior to a QMAX process. For example, in the case that beads are sprayed on a plate as spacers, where beads are landed at random locations of the plate, the inter-spacer distance is not predetermined. Another example of not predetermined inter spacer distance is that the spacers moves during a QMAX processes.

The term “a spacer is fixed on its respective plate” in a QMAX process means that the spacer is attached to a location of a plate and the attachment to that location is maintained during a QMAX (i.e. the location of the spacer on respective plate does not change) process. An example of “a spacer is fixed with its respective plate” is that a spacer is monolithically made of one piece of material of the plate, and the location of the spacer relative to the plate surface does not change during the QMAX process. An example of “a spacer is not fixed with its respective plate” is that a spacer is glued to a plate by an adhesive, but during a use of the plate, during the QMAX process, the adhesive cannot hold the spacer at its original location on the plate surface and the spacer moves away from its original location on the plate surface.

In some embodiments, human hands can be used to press the plates into a closed configuration; In some embodiments, human hands can be used to press the sample into a thin layer. The manners in which hand pressing is employed are described and/or summarized in PCT Application (designating U.S.) Nos. PCT/US2016/045437 filed on Aug. 10, 2016 and PCT/US0216/051775 filed on Sep. 14, 2016, and in US Provisional Application Nos. 62/431,639 filed on Dec. 9, 2016, 62/456,287 filed on Feb. 8, 2017, 62/456,065 filed on Feb. 7, 2017, 62/456,504 filed on Feb. 8, 2017, and 62/460,062 filed on Feb. 16, 2017, which are all hereby incorporated by reference by their entireties.

In some embodiments, human hand can be used to manipulate or handle the plates of the QMAX device. In certain embodiments, the human hand can be used to apply an imprecise force to compress the plates from an open configuration to a closed configuration. In certain embodiments, the human hand can be used to apply an imprecise force to achieve high level of uniformity in the thickness of the sample (e.g. less than 5%, 10%, 15%, or 20% variability).

### (4) Hinges, Opening Notches, Recessed Edge and Sliders

The devices/apparatus, systems, and methods herein disclosed can include or use Q-cards for sample detection, analysis, and quantification. In some embodiments, the Q-card comprises hinges, notches, recesses, and sliders, which help to facilitate the manipulation of the Q card and the measurement of the samples. The structure, material, function, variation and dimension of the hinges, notches, recesses, and sliders are herein disclosed, listed, described, and/or summarized in PCT Application (designating U.S.) Nos. PCT/US2016/045437 and PCT/US0216/051775, which were respectively filed on Aug. 10, 2016 and Sep. 14, 2016, U.S. Provisional Application No. 62/431,639, which was filed on Dec. 9, 2016, U.S. Provisional Application No. 62/456,065, which was filed on Feb. 7, 2017, US Provisional Application Nos. 62/456,287 and 62/456,504, which was filed on Feb. 8, 2017, and U.S. Provisional Application No. 62/539,660, which was filed on Aug. 1, 2017, all of which applications are incorporated herein in their entireties for all purposes.

In some embodiments, the QMAX device comprises opening mechanisms such as but not limited to notches on plate edges or strips attached to the plates, making is easier for a user to manipulate the positioning of the plates, such as but not limited to separating the plates of by hand.

In some embodiments, the QMAX device comprises trenches on one or both of the plates. In certain embodiments, the trenches limit the flow of the sample on the plate.

### (5) Q-Card and Adaptor

The devices/apparatus, systems, and methods herein disclosed can include or use Q-cards for sample detection, analysis, and quantification. In some embodiments, the Q-card is used together with an adaptor that is configured to accommodate the Q-card and connect to a mobile device so that the sample in the Q-card can be imaged, analyzed, and/or measured by the mobile device. The structure, material, function, variation, dimension and connection of the Q-card, the adaptor, and the mobile are herein disclosed, listed, described, and/or summarized in PCT Application (designating U.S.) Nos. PCT/US2016/045437 and PCT/US0216/051775, which were respectively filed on Aug. 10, 2016 and Sep. 14, 2016, U.S. Provisional Application No. 62/456,065, which was filed on Feb. 7, 2017, U.S. Provisional Application Nos. 62/456,287 and 62/456,590, which were filed on Feb. 8, 2017, U.S. Provisional Application No. 62/456,504, which was filed on Feb. 8, 2017, U.S. Provisional Application No. 62/459,544, which was filed on Feb. 15, 2017, and U.S. Provisional Application Nos. 62/460,075 and 62/459,920, which were filed on Feb. 16, 2017, all of which applications are incorporated herein in their entireties for all purposes.

In some embodiments, the adaptor comprises a receptacle slot, which is configured to accommodate the QMAX device when the device is in a closed configuration. In certain embodiments, the QMAX device has a sample deposited therein and the adaptor can be connected to a mobile device (e.g. a smartphone) so that the sample can be read by the mobile device. In certain embodiments, the mobile device can detect and/or analyze a signal from the sample. In certain embodiments, the mobile device can capture images of the sample when the sample is in the QMAX device and positioned in the field of view (FOV) of a camera, which in certain embodiments, is part of the mobile device.

In some embodiments, the adaptor comprises optical components, which are configured to enhance, magnify, and/or optimize the production of the signal from the sample. In some embodiments, the optical components include parts that are configured to enhance, magnify, and/or optimize illumination provided to the sample. In certain embodiments, the illumination is provided by a light source that is part of the mobile device. In some embodiments, the optical components include parts that are configured to enhance, magnify, and/or optimize a signal from the sample.

### (6) Smartphone Detection System

The devices/apparatus, systems, and methods herein disclosed can include or use Q-cards for sample detection, analysis, and quantification. In some embodiments, the Q-card is used together with an adaptor that can connect the Q-card with a smartphone detection system. In some embodiments, the smartphone comprises a camera and/or an illumination source The smartphone detection system, as well the associated hardware and software are herein disclosed, listed, described, and/or summarized in PCT Application (designating U.S.) Nos. PCT/US2016/045437 and PCT/US0216/051775, which were respectively filed on Aug. 10, 2016 and Sep. 14, 2016, U.S. Provisional Application No. 62/456,065, which was filed on Feb. 7, 2017, U.S. Provisional Application Nos. 62/456,287 and 62/456,590, which were filed on Feb. 8, 2017, U.S. Provisional Application No. 62/456,504, which was filed on Feb. 8, 2017, U.S. Provisional Application No. 62/459,544, which was filed on Feb. 15, 2017, and U.S. Provisional Application Nos. 62/460,075 and 62/459,920, which were filed on Feb. 16, 2017, all of which applications are incorporated herein in their entireties for all purposes.

In some embodiments, the smartphone comprises a camera, which can be used to capture images or the sample when the sample is positioned in the field of view of the camera (e.g. by an adaptor). In certain embodiments, the camera includes one set of lenses (e.g. as in iPhone™6). In certain embodiments, the camera includes at least two sets of lenses (e.g. as in iPhone™7). In some embodiments, the smartphone comprises a camera, but the camera is not used for image capturing.

In some embodiments, the smartphone comprises a light source such as but not limited to LED (light emitting diode). In certain embodiments, the light source is used to provide illumination to the sample when the sample is positioned in the field of view of the camera (e.g. by an adaptor). In some embodiments, the light from the light source is enhanced, magnified, altered, and/or optimized by optical components of the adaptor.

In some embodiments, the smartphone comprises a processor that is configured to process the information from the sample. The smartphone includes software instructions that, when executed by the processor, can enhance, magnify, and/or optimize the signals (e.g. images) from the sample. The processor can include one or more hardware components, such as a central processing unit (CPU), an application-specific integrated circuit (ASIC), an application-specific instruction-set processor (ASIP), a graphics processing unit (GPU), a physics processing unit (PPU), a digital signal processor (DSP), a field-programmable gate array (FPGA), a programmable logic device (PLD), a controller, a microcontroller unit, a reduced instruction-set computer (RISC), a microprocessor, or the like, or any combination thereof.

In some embodiments, the smartphone comprises a communication unit, which is configured and/or used to transmit data and/or images related to the sample to another device. Merely by way of example, the communication unit can use a cable network, a wireline network, an optical fiber network, a telecommunications network, an intranet, the Internet, a local area network (LAN), a wide area network (WAN), a wireless local area network (WLAN), a metropolitan area network (MAN), a wide area network (WAN), a public telephone switched network (PSTN), a Bluetooth network, a ZigBee network, a near field communication (NFC) network, or the like, or any combination thereof.

In some embodiments, the smartphone is an iPhone™, an Android™ phone, or a Windows™ phone.

### (7) Detection Methods

The devices/apparatus, systems, and methods herein disclosed can include or be used in various types of detection methods. The detection methods are herein disclosed, listed, described, and/or summarized in PCT Application (designating U.S.) Nos. PCT/US2016/045437 and PCT/US0216/051775, which were respectively filed on Aug. 10, 2016 and Sep. 14, 2016, U.S. Provisional Application No. 62/456,065, which was filed on Feb. 7, 2017, U.S. Provisional Application Nos. 62/456,287, 62/456,528, 62/456631, 62/456522, 62/456598, 62/456603, and 62/456,628, which were filed on Feb. 8, 2017, U.S. Provisional Application Nos. 62/459,276, 62/456,904, 62/457075, and 62/457,009, which were filed on Feb. 9, 2017, and U.S. Provisional Application Nos. 62/459,303, 62/459,337, and 62/459,598, which were filed on Feb. 15, 2017, and U.S. Provisional Application Nos. 62/460,083, 62/460,076, which were filed on Feb. 16, 2017, all of which applications are incorporated herein in their entireties for all purposes.

### (8) Labels, Capture Agent and Detection Agent

The devices/apparatus, systems, and methods herein disclosed can employ various types of labels, capture agents, and detection agents that are used for analytes detection. The labels are herein disclosed, listed, described, and/or summarized in PCT Application (designating U.S.) Nos. PCT/US2016/045437 and PCT/US0216/051775, which were respectively filed on Aug. 10, 2016 and Sep. 14, 2016, U.S. Provisional Application No. 62/456,065, which was filed on Feb. 7, 2017, U.S. Provisional Application No. 62/456,287, which was filed on Feb. 8, 2017, and U.S. Provisional Application No. 62/456,504, which was filed on Feb. 8, 2017, all of which applications are incorporated herein in their entireties for all purposes.

In some embodiments, the label is optically detectable, such as but not limited to a fluorescence label. In some embodiments, the labels include, but are not limited to, IRDye800CW, Alexa 790, Dylight 800, fluorescein, fluorescein isothiocyanate, succinimidyl esters of carboxyfluorescein, succinimidyl esters of fluorescein, 5-isomer of fluorescein dichlorotriazine, caged carboxyfluorescein-alanine-carboxamide, Oregon Green 488, Oregon Green 514; Lucifer Yellow, acridine Orange, rhodamine, tetramethylrhodamine, Texas Red, propidium iodide, JC-1 (5,5′,6,6′-tetrachloro-1,1′,3,3′-tetraethylbenzimidazoylcarbocyanine iodide), tetrabromorhodamine 123, rhodamine 6G, TMRM (tetramethyl rhodamine methyl ester), TMRE (tetramethyl rhodamine ethyl ester), tetramethylrosamine, rhodamine B and 4-dimethylaminotetramethylrosamine, green fluorescent protein, blue-shifted green fluorescent protein, cyan-shifted green fluorescent protein, red-shifted green fluorescent protein, yellow-shifted green fluorescent protein, 4-acetamido-4′-isothiocyanatostilbene-2,2′disulfonic acid; acridine and derivatives, such as acridine, acridine isothiocyanate; 5-(2′-aminoethyl)aminonaphthalene-1-sulfonic acid (EDANS); 4-amino-N-[3-vinylsulfonyl)phenyl]naphth-alimide-3,5 disulfonate; N-(4-anilino-1-naphthyl)maleimide; anthranilamide; 4,4-difluoro-5-(2-thienyl)-4-bora-3a,4a diaza-5-indacene-3-propioni-c acid BODIPY; cascade blue; Brilliant Yellow; coumarin and derivatives: coumarin, 7-amino-4-methylcoumarin (AMC, Coumarin 120), 7-amino-4-trifluoromethylcoumarin (Coumarin 151); cyanine dyes; cyanosine; 4′,6-diaminidino-2-phenylindole (DAPI); 5′,5″-dibromopyrogallol-sulfonaphthalein (Bromopyrogallol Red); 7-diethylamino-3-(4′-isothiocyanatophenyl)-4-methylcoumarin; diethylenetriaamine pentaacetate; 4,4′-diisothiocyanatodihydro-stilbene-2-,2′-disulfonic acid; 4,4′-diisothiocyanatostilbene-2,2′-disulfonic acid; 5-(dimethylaminolnaphthalene-1-sulfonyl chloride (DNS, dansylchloride); 4-dimethylaminophenylazophenyl-4′-isothiocyanate (DABITC); eosin and derivatives: eosin, eosin isothiocyanate, erythrosin and derivatives: erythrosin B, erythrosin, isothiocyanate; ethidium; fluorescein and derivatives: 5-carboxyfluorescein (FAM),5-(4,6-dichlorotriazin-2-yl)amino- -fluorescein (DTAF), 2′,7′dimethoxy-4′5′-dichloro-6-carboxyfluorescein (JOE), fluorescein, fluorescein isothiocyanate, QFITC, (XRITC); fluorescamine; IR144; IR1446; Malachite Green isothiocyanate; 4-methylumbelli-feroneortho cresolphthalein; nitrotyrosine; pararosaniline; Phenol Red; B-phycoerythrin; o-phthaldialdehyde; pyrene and derivatives: pyrene, pyrene butyrate, succinimidyl 1-pyrene; butyrate quantum dots; Reactive Red 4 (Cibacron™ Brilliant Red 3B-A) rhodamine and derivatives: 6-carboxy-X-rhodamine (ROX), 6-carboxyrhodamine (R6G), lissamine rhodamine B sulfonyl chloride rhodamine (Rhod), rhodamine B, rhodamine 123, rhodamine X isothiocyanate, sulforhodamine B, sulforhodamine 101, sulfonyl chloride derivative of sulforhodamine 101 (Texas Red); N,N,N′,N′-tetramethyl-6-carboxyrhodamine (TAMRA); tetramethyl rhodamine; tetramethyl hodamine isothiocyanate (TRITC); riboflavin; 5-(2′-aminoethyl) aminonaphthalene-1-sulfonic acid (EDANS), 4-(4′-dimethylaminophenylazo)benzoic acid (DABCYL), rosolic acid; CAL Fluor Orange 560; terbium chelate derivatives; Cy 3; Cy 5; Cy 5.5; Cy 7; IRD 700; IRD 800; La Jolla Blue; phthalo cyanine; and naphthalo cyanine, coumarins and related dyes, xanthene dyes such as rhodols, resorufins, bimanes, acridines, isoindoles, dansyl dyes, aminophthalic hydrazides such as luminol, and isoluminol derivatives, aminophthalimides, aminonaphthalimides, aminobenzofurans, aminoquinolines, dicyanohydroquinones, fluorescent europium and terbium complexes; combinations thereof, and the like. Suitable fluorescent proteins and chromogenic proteins include, but are not limited to, a green fluorescent protein (GFP), including, but not limited to, a GFP derived from Aequoria victoria or a derivative thereof, e.g., a “humanized” derivative such as Enhanced GFP; a GFP from another species such as Renilla reniformis, Renilla mulleri, or Ptilosarcus guernyi; “humanized” recombinant GFP (hrGFP); any of a variety of fluorescent and colored proteins from Anthozoan species; combinations thereof; and the like.

In any embodiment, the QMAX device can contain a plurality of capture agents and/or detection agents that each bind to a biomarker selected from Tables B1, B2, B3 and/or B7 in U.S. Provisional Application No. 62/234,538 and/or PCT Application No. PCT/US2016/054025, wherein the reading step d) includes obtaining a measure of the amount of the plurality of biomarkers in the sample, and wherein the amount of the plurality of biomarkers in the sample is diagnostic of a disease or condition.

In any embodiment, the capture agent and/or detection agents can be an antibody epitope and the biomarker can be an antibody that binds to the antibody epitope. In some embodiments, the antibody epitope includes a biomolecule, or a fragment thereof, selected from Tables B4, B5 or B6 in U.S. Provisional Application No. 62/234,538 and/or PCT Application No. PCT/US2016/054025. In some embodiments, the antibody epitope includes an allergen, or a fragment thereof, selected from Table B5. In some embodiments, the antibody epitope includes an infectious agent-derived biomolecule, or a fragment thereof, selected from Table B6 in U.S. Provisional Application No. 62/234,538 and/or PCT Application No. PCT/US2016/054025.

In any embodiment, the QMAX device can contain a plurality of antibody epitopes selected from Tables B4, B5 and/or B6 in U.S. Provisional Application No. 62/234,538 and/or PCT Application No. PCT/US2016/054025, wherein the reading step d) includes obtaining a measure of the amount of a plurality of epitope-binding antibodies in the sample, and wherein the amount of the plurality of epitope-binding antibodies in the sample is diagnostic of a disease or condition.

### (9) Analytes

The devices/apparatus, systems, and methods herein disclosed can be applied to manipulation and detection of various types of analytes (including biomarkers). The analytes are herein disclosed, listed, described, and/or summarized in PCT Application (designating U.S.) Nos. PCT/US2016/045437 and PCT/US0216/051775, which were respectively filed on Aug. 10, 2016 and Sep. 14, 2016, U.S. Provisional Application No. 62/456,065, which was filed on Feb. 7, 2017, U.S. Provisional Application No. 62/456,287, which was filed on Feb. 8, 2017, and U.S. Provisional Application No. 62/456,504, which was filed on Feb. 8, 2017, all of which applications are incorporated herein in their entireties for all purposes.

The devices, apparatus, systems, and methods herein disclosed can be used for the detection, purification and/or quantification of various analytes. In some embodiments, the analytes are biomarkers that associated with various diseases. In some embodiments, the analytes and/or biomarkers are indicative of the presence, severity, and/or stage of the diseases. The analytes, biomarkers, and/or diseases that can be detected and/or measured with the devices, apparatus, systems, and/or method of the present invention include the analytes, biomarkers, and/or diseases listed, described and/or summarized in PCT Application (designating U.S.) Nos. PCT/US2016/045437 filed on Aug. 10, 2016, and PCT Application No. PCT/US2016/054025 filed on Sep. 27, 2016, and U.S. Provisional Application Nos. 62/234,538 filed on Sep. 29, 2015, 62/233,885 filed on Sep. 28, 2015, 62/293,188 filed on Feb. 9, 2016, and 62/305,123 filed on Mar. 8, 2016, which are all hereby incorporated by reference by their entireties. For example, the devices, apparatus, systems, and methods herein disclosed can be used in (a) the detection, purification and quantification of chemical compounds or biomolecules that correlates with the stage of certain diseases, e.g., infectious and parasitic disease, injuries, cardiovascular disease, cancer, mental disorders, neuropsychiatric disorders and organic diseases, e.g., pulmonary diseases, renal diseases, (b) the detection, purification and quantification of microorganism, e.g., virus, fungus and bacteria from environment, e.g., water, soil, or biological samples, e.g., tissues, bodily fluids, (c) the detection, quantification of chemical compounds or biological samples that pose hazard to food safety or national security, e.g. toxic waste, anthrax, (d) quantification of vital parameters in medical or physiological monitor, e.g., glucose, blood oxygen level, total blood count, (e) the detection and quantification of specific DNA or RNA from biosamples, e.g., cells, viruses, bodily fluids, (f) the sequencing and comparing of genetic sequences in DNA in the chromosomes and mitochondria for genome analysis or (g) to detect reaction products, e.g., during synthesis or purification of pharmaceuticals.

In some embodiments, the analyte can be a biomarker, an environmental marker, or a foodstuff marker. The sample in some instances is a liquid sample, and can be a diagnostic sample (such as saliva, serum, blood, sputum, urine, sweat, lacrima, semen, or mucus); an environmental sample obtained from a river, ocean, lake, rain, snow, sewage, sewage processing runoff, agricultural runoff, industrial runoff, tap water or drinking water; or a foodstuff sample obtained from tap water, drinking water, prepared food, processed food or raw food.

In any embodiment, the sample can be a diagnostic sample obtained from a subject, the analyte can be a biomarker, and the measured the amount of the analyte in the sample can be diagnostic of a disease or a condition.

In any embodiment, the devices, apparatus, systems, and methods in the present invention can further include diagnosing the subject based on information including the measured amount of the biomarker in the sample. In some cases, the diagnosing step includes sending data containing the measured amount of the biomarker to a remote location and receiving a diagnosis based on information including the measurement from the remote location.

In any embodiment, the biomarker can be selected from Tables B1, 2, 3 or 7 as disclosed in U.S. Provisional Application Nos. 62/234,538, 62/293,188, and/or 62/305,123, and/or PCT Application No. PCT/US2016/054,025, which are all incorporated in their entireties for all purposes. In some instances, the biomarker is a protein selected from Tables B1, 2, or 3. In some instances, the biomarker is a nucleic acid selected from Tables B2, 3 or 7. In some instances, the biomarker is an infectious agent-derived biomarker selected from Table B2. In some instances, the biomarker is a microRNA (miRNA) selected from Table B7.

In any embodiment, the applying step b) can include isolating miRNA from the sample to generate an isolated miRNA sample, and applying the isolated miRNA sample to the disk-coupled dots-on-pillar antenna (QMAX device) array.

In any embodiment, the QMAX device can contain a plurality of capture agents that each bind to a biomarker selected from Tables B1, B2, B3 and/or B7, wherein the reading step d) includes obtaining a measure of the amount of the plurality of biomarkers in the sample, and wherein the amount of the plurality of biomarkers in the sample is diagnostic of a disease or condition.

In any embodiment, the capture agent can be an antibody epitope and the biomarker can be an antibody that binds to the antibody epitope. In some embodiments, the antibody epitope includes a biomolecule, or a fragment thereof, selected from Tables B4, B5 or B6. In some embodiments, the antibody epitope includes an allergen, or a fragment thereof, selected from Table B5. In some embodiments, the antibody epitope includes an infectious agent-derived biomolecule, or a fragment thereof, selected from Table B6.

In any embodiment, the QMAX device can contain a plurality of antibody epitopes selected from Tables B4, B5 and/or B6, wherein the reading step d) includes obtaining a measure of the amount of a plurality of epitope-binding antibodies in the sample, and wherein the amount of the plurality of epitope-binding antibodies in the sample is diagnostic of a disease or condition.

In any embodiment, the sample can be an environmental sample, and wherein the analyte can be an environmental marker. In some embodiments, the environmental marker is selected from Table B8 in U.S. Provisional Application No. 62/234,538 and/or PCT Application No. PCT/US2016/054025.

In any embodiment, the method can include receiving or providing a report that indicates the safety or harmfulness for a subject to be exposed to the environment from which the sample was obtained.

In any embodiment, the method can include sending data containing the measured amount of the environmental marker to a remote location and receiving a report that indicates the safety or harmfulness for a subject to be exposed to the environment from which the sample was obtained.

In any embodiment, the QMAX device array can include a plurality of capture agents that each binds to an environmental marker selected from Table B8, and wherein the reading step d) can include obtaining a measure of the amount of the plurality of environmental markers in the sample.

In any embodiment, the sample can be a foodstuff sample, wherein the analyte can be a foodstuff marker, and wherein the amount of the foodstuff marker in the sample can correlate with safety of the foodstuff for consumption. In some embodiments, the foodstuff marker is selected from Table B9.

In any embodiment, the method can include receiving or providing a report that indicates the safety or harmfulness for a subject to consume the foodstuff from which the sample is obtained.

In any embodiment, the method can include sending data containing the measured amount of the foodstuff marker to a remote location and receiving a report that indicates the safety or harmfulness for a subject to consume the foodstuff from which the sample is obtained.

In any embodiment, the devices, apparatus, systems, and methods herein disclosed can include a plurality of capture agents that each binds to a foodstuff marker selected from Table B9 from in U.S. Provisional Application No. 62/234,538 and PCT Application No. PCT/US2016/054025, wherein the obtaining can include obtaining a measure of the amount of the plurality of foodstuff markers in the sample, and wherein the amount of the plurality of foodstuff marker in the sample can correlate with safety of the foodstuff for consumption.

Also provided herein are kits that find use in practicing the devices, systems and methods in the present invention.

The amount of sample can be about a drop of a sample. The amount of sample can be the amount collected from a pricked finger or fingerstick. The amount of sample can be the amount collected from a microneedle or a venous draw.

A sample can be used without further processing after obtaining it from the source, or can be processed, e.g., to enrich for an analyte of interest, remove large particulate matter, dissolve or resuspend a solid sample, etc.

Any suitable method of applying a sample to the QMAX device can be employed. Suitable methods can include using a pipet, dropper, syringe, etc. In certain embodiments, when the QMAX device is located on a support in a dipstick format, as described below, the sample can be applied to the QMAX device by dipping a sample-receiving area of the dipstick into the sample.

A sample can be collected at one time, or at a plurality of times. Samples collected over time can be aggregated and/or processed (by applying to a QMAX device and obtaining a measurement of the amount of analyte in the sample, as described herein) individually. In some instances, measurements obtained over time can be aggregated and can be useful for longitudinal analysis over time to facilitate screening, diagnosis, treatment, and/or disease prevention.

Washing the QMAX device to remove unbound sample components can be done in any convenient manner, as described above. In certain embodiments, the surface of the QMAX device is washed using binding buffer to remove unbound sample components.

Detectable labeling of the analyte can be done by any convenient method. The analyte can be labeled directly or indirectly. In direct labeling, the analyte in the sample is labeled before the sample is applied to the QMAX device. In indirect labeling, an unlabeled analyte in a sample is labeled after the sample is applied to the QMAX device to capture the unlabeled analyte, as described below.

### (10) Applications

The devices/apparatus, systems, and methods herein disclosed can be used for various applications (fields and samples). The applications are herein disclosed, listed, described, and/or summarized in PCT Application (designating U.S.) Nos. PCT/US2016/045437 and PCT/US0216/051775, which were respectively filed on Aug. 10, 2016 and Sep. 14, 2016, U.S. Provisional Application No. 62/456,065, which was filed on Feb. 7, 2017, U.S. Provisional Application No. 62/456,287, which was filed on Feb. 8, 2017, and U.S. Provisional Application No. 62/456,504, which was filed on Feb. 8, 2017, all of which applications are incorporated herein in their entireties for all purposes.

In some embodiments, the devices, apparatus, systems, and methods herein disclosed are used in a variety of different application in various field, wherein determination of the presence or absence, quantification, and/or amplification of one or more analytes in a sample are desired. For example, in certain embodiments the subject devices, apparatus, systems, and methods are used in the detection of proteins, peptides, nucleic acids, synthetic compounds, inorganic compounds, organic compounds, bacteria, virus, cells, tissues, nanoparticles, and other molecules, compounds, mixtures and substances thereof. The various fields in which the subject devices, apparatus, systems, and methods can be used include, but are not limited to: diagnostics, management, and/or prevention of human diseases and conditions, diagnostics, management, and/or prevention of veterinary diseases and conditions, diagnostics, management, and/or prevention of plant diseases and conditions, agricultural uses, veterinary uses, food testing, environments testing and decontamination, drug testing and prevention, and others.

The applications of the present invention include, but are not limited to: (a) the detection, purification, quantification, and/or amplification of chemical compounds or biomolecules that correlates with certain diseases, or certain stages of the diseases, e.g., infectious and parasitic disease, injuries, cardiovascular disease, cancer, mental disorders, neuropsychiatric disorders and organic diseases, e.g., pulmonary diseases, renal diseases, (b) the detection, purification, quantification, and/or amplification of cells and/or microorganism, e.g., virus, fungus and bacteria from the environment, e.g., water, soil, or biological samples, e.g., tissues, bodily fluids, (c) the detection, quantification of chemical compounds or biological samples that pose hazard to food safety, human health, or national security, e.g. toxic waste, anthrax, (d) the detection and quantification of vital parameters in medical or physiological monitor, e.g., glucose, blood oxygen level, total blood count, (e) the detection and quantification of specific DNA or RNA from biological samples, e.g., cells, viruses, bodily fluids, (f) the sequencing and comparing of genetic sequences in DNA in the chromosomes and mitochondria for genome analysis or (g) the detection and quantification of reaction products, e.g., during synthesis or purification of pharmaceuticals.

In some embodiments, the subject devices, apparatus, systems, and methods are used in the detection of nucleic acids, proteins, or other molecules or compounds in a sample. In certain embodiments, the devices, apparatus, systems, and methods are used in the rapid, clinical detection and/or quantification of one or more, two or more, or three or more disease biomarkers in a biological sample, e.g., as being employed in the diagnosis, prevention, and/or management of a disease condition in a subject. In certain embodiments, the devices, apparatus, systems, and methods are used in the detection and/or quantification of one or more, two or more, or three or more environmental markers in an environmental sample, e.g. sample obtained from a river, ocean, lake, rain, snow, sewage, sewage processing runoff, agricultural runoff, industrial runoff, tap water or drinking water. In certain embodiments, the devices, apparatus, systems, and methods are used in the detection and/or quantification of one or more, two or more, or three or more foodstuff marks from a food sample obtained from tap water, drinking water, prepared food, processed food or raw food.

In some embodiments, the subject device is part of a microfluidic device. In some embodiments, the subject devices, apparatus, systems, and methods are used to detect a fluorescence or luminescence signal. In some embodiments, the subject devices, apparatus, systems, and methods include, or are used together with, a communication device, such as but not limited to: mobile phones, tablet computers and laptop computers. In some embodiments, the subject devices, apparatus, systems, and methods include, or are used together with, an identifier, such as but not limited to an optical barcode, a radio frequency ID tag, or combinations thereof.

In some embodiments, the sample is a diagnostic sample obtained from a subject, the analyte is a biomarker, and the measured amount of the analyte in the sample is diagnostic of a disease or a condition. In some embodiments, the subject devices, systems and methods further include receiving or providing to the subject a report that indicates the measured amount of the biomarker and a range of measured values for the biomarker in an individual free of or at low risk of having the disease or condition, wherein the measured amount of the biomarker relative to the range of measured values is diagnostic of a disease or condition.

In some embodiments, the sample is an environmental sample, and wherein the analyte is an environmental marker. In some embodiments, the subject devices, systems and methods includes receiving or providing a report that indicates the safety or harmfulness for a subject to be exposed to the environment from which the sample was obtained. In some embodiments, the subject devices, systems and methods include sending data containing the measured amount of the environmental marker to a remote location and receiving a report that indicates the safety or harmfulness for a subject to be exposed to the environment from which the sample was obtained.

In some embodiments, the sample is a foodstuff sample, wherein the analyte is a foodstuff marker, and wherein the amount of the foodstuff marker in the sample correlate with safety of the foodstuff for consumption. In some embodiments, the subject devices, systems and methods include receiving or providing a report that indicates the safety or harmfulness for a subject to consume the foodstuff from which the sample is obtained. In some embodiments, the subject devices, systems and methods include sending data containing the measured amount of the foodstuff marker to a remote location and receiving a report that indicates the safety or harmfulness for a subject to consume the foodstuff from which the sample is obtained.

### (11) Dimensions

The devices, apparatus, systems, and methods herein disclosed can include or use a QMAX device, which can comprise plates and spacers. In some embodiments, the dimension of the individual components of the QMAX device and its adaptor are listed, described and/or summarized in PCT Application (designating U.S.) No. PCT/US2016/045437 filed on Aug. 10, 2016, and U.S. Provisional Application Nos. 62,431,639 filed on Dec. 9, 2016 and 62/456,287 filed on Feb. 8, 2017, which are all hereby incorporated by reference by their entireties.

In some embodiments, the dimensions are listed in the Tables below:

Plates

| Parameters | Embodiments | Preferred Embodiments |
|---|---|---|
| Shape | round, ellipse, rectangle, triangle, polygonal, ring- | at least one of the two (or more) plates |
| | shaped, or any superposition of these shapes; the | of the QMAX card has round corners for user |
| | two (or more) plates of the QMAX card can have | safety concerns, wherein the round corners |
| | the same size and/or shape, or different size | have a diameter of 100 um or less, 200 um or |
| | and/or shape; | less, 500 um or less, 1 mm or less, 2 mm or |
| | | less, 5 mm or less, 10 mm or less, 50 mm or less, |
| | | or in a range between any two of the values. |
| Thickness | the average thickness for at least one of the plates | For at least one of the plates is |
| | is 2 nm or less, 10 nm or less, 100 nm or less, 200 nm | in the range of 0.5 to 1.5 mm; around 1 |
| | or less, 500 nm or less, 1000 nm or less, 2 μm | mm; in the range of 0.15 to 0.2 mm; or |
| | (micron) or less, 5 μm or less, 10 μm or less, 20 | around 0.175 mm |
| | μm or less, 50 μm or less, 100 μm or less, 150 μm | |
| | or less, 200 μm or less, 300 μm or less, 500 μm or | |
| | less, 800 μm or less, 1 mm (millimeter) or less, 2 | |
| | mm or less, 3 mm or less, 5 mm or less, 10 mm or | |
| | less, 20 mm or less, 50 mm or less, 100 mm or | |
| | less, 500 mm or less, or in a range between any | |
| | two of these values | |
| Lateral Area | For at least one of the plate is 1 mm2 (square | For at least one plate of the QMAX |
| | millimeter) or less, 10 mm2 or less, 25 mm2 or less, | card is in the range of 500 to |
| | 50 mm2 or less, 75 mm2 or less, 1 cm2 (square | 1000 mm²; or around 750 mm². |
| | centimeter) or less, 2 cm2 or less, 3 cm2 or less, 4 | |
| | cm2 or less, 5 cm2 or less, 10 cm2 or less, 100 cm2 | |
| | or less, 500 cm2 or less, 1000 cm2 or less, 5,000 | |
| | cm2 or less, 10,000 cm2 or less, 10,000 cm2 or less, | |
| | or in a range between any two of these values | |
| Lateral Linear | For at least one of the plates of the QMAX card is 1 | For at least one plate of the |
| Dimension (width, | mm or less, 5 mm or less, 10 mm or less, 15 mm or | QMAX card is in the range of |
| length, or | less, 20 mm or less, 25 mm or less, 30 mm or less, | 20 to 30 mm; or around 24 mm |
| diameter, etc.) | 35 mm or less, 40 mm or less, 45 mm or less, 50 | |
| | mm or less, 100 mm or less, 200 mm or less, 500 | |
| | mm or less, 1000 mm or less, 5000 mm or less, or | |
| | in a range between any two of these values | |
| Recess width | 1 um or less, 10 um or less, 20 um or less, 30 um | In the range of 1 mm to |
| | or less, 40 um or less, 50 um or less, 100 um or | 10 mm; Or About 5 mm |
| | less, 200 um or less, 300 um or less, 400 um or | |
| | less, 500 um or less, 7500 um or less, 1 mm or | |
| | less, 5 mm or less, 10 mm or less, 100 mm or less, | |
| | or 1000 mm or less, or in a range between any | |
| | two of these values. | |

Hinge

| Parameters | Embodiments | Preferred Embodiments |
|---|---|---|
| Number | 1, 2, 3, 4, 5, or more | 1 or 2 |
| Length of | 1 mm or less, 2 mm or less, 3 mm or less, 4 mm or less, 5 mm or less, 10 mm or less, 15 mm or | In the range of |
| Hinge Joint | less, 20 mm or less, 25 mm or less, 30 mm or less, 40 mm or less, 50 mm or less, 100 mm or | 5 mm to 30 mm. |
| | less, 200 mm or less, or 500 mm or less, or in a range between any two of these values | |
| Ratio (hinge | 1.5 or less, 1 or less, 0.9 or less, 0.8 or less, 0.7 or less, 0.6 or less, 0.5 or less, 0.4 or less, 0.3 or | In the range of 0.2 |
| joint length | less, 0.2 or less, 0.1 or less, 0.05 or less or in a range between any two of these values. | to 1; or about 1 |
| vs. aligning | | |
| plate edge | | |
| length | | |
| Area | 1 mm² or less, 5 mm² or less, 10 mm² or less, 20 mm² or less, 30 mm² or less, 40 mm² or less, 50 | In the range of 20 |
| | mm² or less, 100 mm² or less, 200 mm² or less, 500 mm² or less, or in a range between any of the | to 200 mm²; or about |
| | two values | 120 mm² |
| Ratio (hinge | 1 or less, 0.9 or less, 0.8 or less, 0.7 or less, 0.6 or less, 0.5 or less, 0.4 or less, 0.3 or less, 0.2 or less | In the range of 0.05 |
| area vs. | 0.1 or less, 0.05 or less, 0.01 or less or in a range between any two of these values | to 0.2, around 0.15 |
| plate area) | | |
| Max. Open | 15 or less, 30 or less, 45 or less, 60 or less, 75 or less, 90 or less, 105 or less, 120 or less, 135 or | In the range of 90 to |
| Degree | less, 150 or less, 165 or less, 180 or less, 195 or less, 210 or less, 225 or less, 240 or less, 255 or | 180 degrees |
| | less, 270 or less, 285 or less, 300 or less, 315 or less, 330 or less, 345 or less or 360 or less degrees, | |
| | or in a range between any two of these values | |
| No. of | 1, 2, 3, 4, 5, or more | 1 or 2 |
| Layers | | |
| Layer | 0.1 um or less, 1 um or less, 2 um or less, 3 um or less, 5 um or | In the range of 20 um |
| thickness | less, 10 um or less, 20 um or less, 30 um or less, 50 um or less, | to 1 mm; or Around |
| | 100 um or less, 200 um or less, 300 um or less, 500 um or | 50 um |
| | less, 1 mm or less, 2 mm or less, and a range between any two of these | |
| | values | |
| Angle- | Limiting the angle adjustment with no more | No more than ±2 |
| maintaining | than ±90, ±45, ±30, ±25, ±20, ±15, ±10, ±8, ±6, ±5, ±4, ±3, ±2, | |
| | or ±1, or in a range between any two of these values | |

Notch

| Parameters | Embodiments | Preferred Embodiments |
|---|---|---|
| Number | 1, 2, 3, 4, 5, or more | 1 or 2 |
| Shape | Round, ellipse, rectangle, triangle, polygon, ring- | Part of a circle |
| | shaped, or any superposition or portion of these | |
| | shapes. | |
| Positioning | Any location along any edge except the hinge | |
| | edge, or any corner joint by non-hinge edges | |
| Lateral Linear | 1 mm or less, 2.5 mm or less, 5 mm or less, 10 | In the range of 5 mm to |
| Dimension (Length | mm or less, 15 mm or less, 20 mm or less, 25 | 15 mm; or about 10 mm |
| along the edge, | mm or less, 30 mm or less, 40 mm or less, 50 | |
| radius, etc.) | mm or less, or in a range between any two of | |
| | these values | |
| Area | 1 mm² (square millimeter) or less, 10 mm² or less, | In the range of 10 to |
| | 25 mm² or less, 50 mm² or less, 75 mm² or less or | 150 mm²; or about 50 mm² |
| | in a range between any two of these values. | |

Trench

| Parameters | Embodiments | Preferred Embodiments |
|---|---|---|
| Number | 1, 2, 3, 4, 5, or more | 1 or 2 |
| Shape | Closed (round, ellipse, rectangle, triangle, | |
| | polygon, ring-shaped, or any superposition or | |
| | portion of these shapes) or open-ended (straight | |
| | line, curved line, arc, branched tree, or any other | |
| | shape with open endings); | |
| Length | 0.001 mm or less, 0.005 mm or less, 0.01 mm or | |
| | less, 0.05 mm or less, 0.1 mm or less, 0.5 mm or | |
| | less, 1 mm or less, 2 mm or less, 5 mm or less, 10 | |
| | mm or less, 20 mm or less, 50 mm or less, 100 | |
| | mm or less, or in a range between any two of | |
| | these values | |
| Cross- | 0.001 mm² or less, 0.005 mm² or less, 0.01 mm² or | |
| sectional | less, 0.05 mm² or less, 0.1 mm² or less, 0.5 mm² or | |
| Area | less, 1 mm² or less, 2 mm² or less, 5 mm² or less, | |
| | 10 mm² or less, 20 mm² or less, or in a range | |
| | between any two of these values. | |
| Volume | 0.1 uL or more, 0.5 uL or more, 1 uL or more, 2 uL | In the range of 1 uL to |
| | or more, 5 uL or more, 10 uL or more, 30 uL or | 20 uL; or About 5 uL |
| | more, 50 uL or more, 100 uL or more, 500 uL or | |
| | more, 1 mL or more, or in a range between any two | |
| | of these values | |

Receptacle Slot

| Parameters | Embodiments | Preferred Embodiments |
|---|---|---|
| Shape of | round, ellipse, rectangle, triangle, polygon, ring- | |
| receiving | shaped, or any superposition of these shapes; | |
| area | | |
| Difference | 100 nm, 500 nm, 1 um, 2 um, 5 um, 10 um, 50 | In the range of 50 to |
| between | um, 100 um, 300 um, 500 um, 1 mm, 2 mm, 5 | 300 um; or about 75 um |
| sliding track | mm, 1 cm, or in a range between any two of the | |
| gap size | values. | |
| and card | | |
| thickness | | |
| Difference | 1 mm² (square millimeter) or less, 10 mm² or less, | |
| between | 25 mm² or less, 50 mm² or less, 75 mm² or less, 1 | |
| receiving | cm² (square centimeter) or less, 2 cm² or less, 3 | |
| area and | cm² or less, 4 cm² or less, 5 cm² or less, 10 cm² or | |
| card area | less, 100 cm² or less, or in a range between any of | |
| | the two values. | |

### (12) Cloud

The devices/apparatus, systems, and methods herein disclosed can employ cloud technology for data transfer, storage, and/or analysis. The related cloud technologies are herein disclosed, listed, described, and/or summarized in PCT Application (designating U.S.) Nos. PCT/US2016/045437 and PCT/US0216/051775, which were respectively filed on Aug. 10, 2016 and Sep. 14, 2016, U.S. Provisional Application No. 62/456,065, which was filed on Feb. 7, 2017, U.S. Provisional Application No. 62/456,287, which was filed on Feb. 8, 2017, and U.S. Provisional Application No. 62/456,504, which was filed on Feb. 8, 2017, all of which applications are incorporated herein in their entireties for all purposes.

In some embodiments, the cloud storage and computing technologies can involve a cloud database. Merely by way of example, the cloud platform can include a private cloud, a public cloud, a hybrid cloud, a community cloud, a distributed cloud, an inter-cloud, a multi-cloud, or the like, or any combination thereof. In some embodiments, the mobile device (e.g. smartphone) can be connected to the cloud through any type of network, including a local area network (LAN) or a wide area network (WAN).

In some embodiments, the data (e.g. images of the sample) related to the sample is sent to the cloud without processing by the mobile device and further analysis can be conducted remotely. In some embodiments, the data related to the sample is processed by the mobile device and the results are sent to the cloud. In some embodiments, both the raw data and the results are transmitted to the cloud.

### Additional Notes

Further examples of inventive subject matter according to the present disclosure are described in the following enumerated paragraphs.

It must be noted that as used herein and in the appended claims, the singular forms “a”, “an”, and “the” include plural referents unless the context clearly dictates otherwise, e.g., when the word “single” is used. For example, reference to “an analyte” includes a single analyte and multiple analytes, reference to “a capture agent” includes a single capture agent and multiple capture agents, reference to “a detection agent” includes a single detection agent and multiple detection agents, and reference to “an agent” includes a single agent and multiple agents.

As used herein, the terms “adapted” and “configured” mean that the element, component, or other subject matter is designed and/or intended to perform a given function. Thus, the use of the terms “adapted” and “configured” should not be construed to mean that a given element, component, or other subject matter is simply “capable of” performing a given function. Similarly, subject matter that is recited as being configured to perform a particular function may additionally or alternatively be described as being operative to perform that function.

As used herein, the phrase, “for example,” the phrase, “as an example,” and/or simply the terms “example” and “exemplary” when used with reference to one or more components, features, details, structures, embodiments, and/or methods according to the present disclosure, are intended to convey that the described component, feature, detail, structure, embodiment, and/or method is an illustrative, non-exclusive example of components, features, details, structures, embodiments, and/or methods according to the present disclosure. Thus, the described component, feature, detail, structure, embodiment, and/or method is not intended to be limiting, required, or exclusive/exhaustive; and other components, features, details, structures, embodiments, and/or methods, including structurally and/or functionally similar and/or equivalent components, features, details, structures, embodiments, and/or methods, are also within the scope of the present disclosure.

As used herein, the phrases “at least one of” and “one or more of,” in reference to a list of more than one entity, means any one or more of the entity in the list of entity, and is not limited to at least one of each and every entity specifically listed within the list of entity. For example, “at least one of A and B” (or, equivalently, “at least one of A or B,” or, equivalently, “at least one of A and/or B”) may refer to A alone, B alone, or the combination of A and B.

As used herein, the term “and/or” placed between a first entity and a second entity means one of (1) the first entity, (2) the second entity, and (3) the first entity and the second entity. Multiple entity listed with “and/or” should be construed in the same manner, i.e., “one or more” of the entity so conjoined. Other entity may optionally be present other than the entity specifically identified by the “and/or” clause, whether related or unrelated to those entities specifically identified.

Where numerical ranges are mentioned herein, the invention includes embodiments in which the endpoints are included, embodiments in which both endpoints are excluded, and embodiments in which one endpoint is included and the other is excluded. It should be assumed that both endpoints are included unless indicated otherwise. Furthermore, unless otherwise indicated or otherwise evident from the context and understanding of one of ordinary skill in the art.

In the event that any patents, patent applications, or other references are incorporated by reference herein and (1) define a term in a manner that is inconsistent with and/or (2) are otherwise inconsistent with, either the non-incorporated portion of the present disclosure or any of the other incorporated references, the non-incorporated portion of the present disclosure shall control, and the term or incorporated disclosure therein shall only control with respect to the reference in which the term is defined and/or the incorporated disclosure was present originally.

As will be apparent to those of skill in the art upon reading this disclosure, each of the individual embodiments described and illustrated herein has discrete components and features which can be readily separated from or combined with the features of any of the other several embodiments without departing from the scope or spirit of the present teachings. Any recited method can be carried out in the order of events recited or in any other order which is logically possible.

One with skill in the art will appreciate that the present invention is not limited in its application to the details of construction, the arrangements of components, category selections, weightings, predetermined signal limits, or the steps set forth in the description or drawings herein. The invention is capable of other embodiments and of being practiced or being carried out in many different ways.

The practice of various embodiments of the present disclosure employs, unless otherwise indicated, conventional techniques of immunology, biochemistry, chemistry, molecular biology, microbiology, cell biology, genomics and recombinant DNA, which are within the skill of the art. See Green and Sambrook, MOLECULAR CLONING: A LABORATORY MANUAL, 4^{th} edition (2012); CURRENT PROTOCOLS IN MOLECULAR BIOLOGY (F. M. Ausubel, et al. eds., (1987)); the series METHODS IN ENZYMOLOGY (Academic Press, Inc.): PCR 2: A PRACTICAL APPROACH (M. J. MacPherson, B. D. Hames and G. R. Taylor eds. (1995)), Harlow and Lane, eds. (1988) ANTIBODIES, A LABORATORY MANUAL, and ANIMAL CELL CULTURE (R. I. Freshney, ed. (1987)).

1. An apparatus for assaying a sample that contains an analyte and interference elements, comprising:
a sample holder that is configured to hold a sample that contains an analyte and one or more interference elements;
an imager and a software that are configured to identify (a) a region in the sample that has less interference element concentration (“interference element poor region”) than another region in the sample (“interference element rich region”); and
a detector that is configured to detect a signal related to the analyte in the interference element poor region and/or in the interference element rich region;
wherein the “interference element” is an element in a sample, wherein the element has an interference with a signal related to an analyte in the sample, wherein the interference refers to blocking, reducing, attenuating, and/or disrupting the signal related to the analyte.

2. An apparatus for assaying a sample that contains an analyte and interference elements, comprising:
a sample holder that is configured to hold a sample that contains an analyte and one or more interference elements; wherein the sample comprising two plates separated by a spacing of 250 um or less, and wherein at least a part of the sample is between the two plates, and
an imager and a software that are configured to identify a region in the sample that has less interference element concentration (“interference element poor region”) than another region in the sample (“interference element rich region”); and
a detector that is configured to detect a signal related to the analyte in the interference element poor region;
wherein the “interference element” is an element in a sample, wherein the element has an interference with a signal related to an analyte in the sample, wherein the interference refers to blocking, reducing, attenuating, and/or disrupting the signal related to the analyte.

3. An apparatus for assaying a liquid sample that contains an analyte and interference elements, comprising:
a sample holder that comprises a first plate and a second plate and is configured to hold a sample that contains an analyte and one or more interference elements, wherein:
i. at least a part of the sample is between the first plate and second plate; and
ii. one or both of the plates are configured to allow the at least a part of the sample visible through the one or both of the plates;
an imager and a software that are configured to identify, in the at least a part of sample, a region that has less interference element concentration (“interference element poor region”) than another region in the sample layer (“interference element rich region”); and
a detector that is configured to detect a signal related to the analyte in an interference element poor region.

4. An apparatus for assaying a liquid sample that contains an analyte and interference elements, comprising:
a sample holder that comprises a first plate, a second plate, and spacers and is configured to hold a sample that contains an analyte and one or more interference elements, wherein:
i. the first plate and second plate moveable relative to each other;
ii. the spacers are fixed on one or both of the plates and have a uniform height;
ii. the first plate and second plate are configured to compress the sample into a layer of uniform thickness that substantially equals the height of the spacers;
an imager and a software that are configured to identify a region in the sample layer that has less interference element concentration (“interference element poor region”) than another region in the sample layer (“interference element rich region”); and
a detector that is configured to detect a signal related to the analyte in the interference element poor region.

5. A kit for assaying a sample that contains an analyte and interference elements, comprising:
the apparatus of claim 1; and
an aggregation reagent that causes or assists a sample to have a region that has less interference element concentration (“interference element poor region”) than another region in the sample (“interference element rich region”).

6. A method for assaying a sample that contains an analyte and interference elements, comprising:
i. obtaining a sample holder;
ii. depositing in the sample holder a sample that contains an analyte and one or more interference elements;
iii. imaging and identifying, with an imager and a software, (a) the regions in the sample that has an inference element concentration (“interference element poor region”) substantially less than that in other region(s) (“interference element rich region”); and
iv. measuring a signal related to the analyte in the interference element rich region and/or in the interference element poor region.

7. A method for assaying a sample that contains an analyte and interference elements, comprising:
i. obtaining a sample holder;
ii. depositing in the sample holder a sample that contains an analyte and one or more interference elements, wherein the sample comprising two plates separated by a spacing of 250 um or less, and wherein at least a part of the sample is between the two plates;
iii. imaging and identifying, with an imager and a software, (a) the regions in the sample that has less inference element concentration (“interference element rich region”) than anther region (“interference element poor region”); and
iv. measuring a signal related to the analyte in the interference element poor region.

8. The method of claim 6, wherein it further comprises adding an aggregation reagent that causes or assists a sample to have a region that has less interference element concentration (“interference element poor region”) than another region in the sample (“interference element rich region”).

9. The method of claim 6, wherein the signal related to analyte in the interference element poor region is measured.

10. The method of claim 6, further comprising calculating a concentration of the analyte in the sample based on the signal related to the analyte in the interference element rich region.

11. The method of claim 6, further comprising calculating a concentration of the analyte in the sample based on the signal related to the analyte in the interference element poor region.

12. The method of claim 6, wherein the interference element poor region has an area that is less than 30%, 20%, 10%, 5%, 1%, or 0.1% covered by the interference element.

13. The method of claim 6, wherein the sample is compressed by the sample holder into a layer of uniform thickness, and the method further comprises:
calculating the volume of the sample based on an area of the sample layer.

14. The method of claim 6, further comprising: calculating the concentration of the analyte in the sample based on the signal related to the analyte in the interference element rich region and/or the interference element poor region, and the volume of the sample.

15. The method of claim 6, further comprising: calculating the concentration of the analyte in the sample based on the signal related to the analyte in the interference element poor region, and the volume of the sample in the interference element poor region.

16. The apparatus of claim 1, wherein the detector is a part or a whole of the imager

17. The apparatus of claim 1, wherein the detector is a separate device from the imager

18. The apparatus of claim 1, wherein the apparatus further comprises an aggregation reagent that causes or assists a sample to have a region that has less interference element concentration (“interference element poor region”) than another region in the sample (“interference element rich region”).

19. The apparatus of claim 18, wherein the aggregation regent is coated on the sample holder.

20. The apparatus of claim 18, wherein the aggregation reagent is coated on the sample holder, and the aggregation reagent is a dry reagent.

21. The apparatus of claim 1, wherein the imager and the software are further configured to identify the interference element rich region.

22. The apparatus of claim 1, wherein the detector is further configured to detect a signal related to the analyte in the interference element rich region.

23. The apparatus of claim 1, wherein the detector is further configured to detect a signal related to the interference elements in the interference element rich region.

24. The apparatus of claim 1, wherein the sample holder is configured to compress the sample into a thin layer.

25. The apparatus of claim 1, wherein the sample holder is configured to compress the sample into a thin layer with uniform thickness.

26. The apparatus of claim 1, wherein the interference element rich region has an area that is at least 30%, 40%, 50%, 60%, 70%, 80%, 90%, or 99% covered by the interference elements.

27. The apparatus of claim 1, wherein the interference element poor region has an area that is less than 30%, 20%, 10%, 5%, 1%, or 0.1% covered by the interference elements.

28. The apparatus of claim 1, wherein the interference rich regions are formed without facilitation of factors not in the sample.

29. The apparatus of claim 1, wherein the interference rich regions are formed with facilitation of factors not in the sample.

30. The apparatus of claim 1, wherein the interference element poor and/or rich regions in the sample form one or more microdomains.

31. The apparatus of claim 30, wherein each of the one or more microdomain has an average dimension of less than 1 um, 10 um, 50 um, 100 um, 200 um, 250 um, 500 um, 600 um, 700 um, or 800 um, or in a range between any of the two values.

32. The apparatus of claim 30, wherein only the interference element poor regions in the sample form one or more microdomains, and wherein a microdomain is an interference element poor or region that has an average dimension of 800 um or less

33. The apparatus of claim 30, wherein only the interference element rich regions in the sample form one or more microdomains, and wherein a microdomain is an interference element poor or region that has an average dimension of 800 um or less.

34. The apparatus of claim 30, wherein each of the one or more microdomain has an average dimension of 700 um or less.

35. The apparatus of claim 30, wherein each of the one or more microdomain has an average dimension of 600 um or less.

36. The apparatus of claim 30, wherein each of the one or more microdomain has an average dimension of 500 um or less.

37. The apparatus of claim 30, wherein each of the one or more microdomain has an average dimension of 250 um or less.

38. The apparatus of claim 30, wherein each of the one or more microdomain has an average dimension of 100 um or less.

39. The apparatus of claim 30, wherein each of the one or more microdomain has an average dimension of 50 um or less.

40. The apparatus of claim 30, wherein each of the one or more microdomain has an average dimension of 10 um or less.

41. The apparatus of claim 30, wherein each of the one or more microdomain has an average dimension of 1 um or less.

42. The apparatus of claim 30, wherein each of the one or more microdomain has an average dimension in the range of 1-800 um, 50-800 um, 100-800 um, 250-800 um, 500-800 um, or 600-800 um.

43. The apparatus of claim 30, wherein each of the one or more microdomain has an average dimension in the range of 1-800 um, 1-700 um, 1-600 um, 1-500 um, 1-250 um, 1-100 um, 1-50 um, 1-25 um, or 1-10 um.

44. The apparatus of claim 1, wherein at least part of the sample is compressed into a thin layer that has an average thickness of 500 um or less, 400 um or less, 300 um or less, 200 um or less, 175 um or less, 150 um or less, 125 um or less, 100 um or less, 75 um or less, 50 um or less, 40 um or less, 30 um or less, 20 um or less, 10 um or less, 5 um or less, 4 um or less, 3 um or less, 2 um or less, 1.8 um or less, 1.5 um or less, 1 um or less, 0.5 um or less, 0.2 um or less, 0.1 um or less, 50 nm or less, 20 nm or less, 10 nm or less, or in a range between any of the two values.

45. The apparatus of claim 1, wherein at least part of the sample is compressed into a thin layer, and wherein for a specific part of the sample that has an average thickness of 500 um or less, 400 um or less, 300 um or less, 200 um or less, 175 um or less, 150 um or less, 125 um or less, 100 um or less, 75 um or less, 50 um or less, 40 um or less, 30 um or less, 20 um or less, 10 um or less, 5 um or less, 4 um or less, 3 um or less, 2 um or less, 1.8 um or less, 1.5 um or less, 1 um or less, 0.5 um or less, 0.2 um or less, 0.1 um or less, 50 nm or less, 20 nm or less, 10 nm or less, or in a range between any of the two values, only the interference rich regions exist.

46. The apparatus of claim 1, wherein at least part of the sample is compressed into a thin layer, wherein for a specific part of the sample that has an average thickness of 500 um or less, 400 um or less, 300 um or less, 200 um or less, 175 um or less, 150 um or less, 125 um or less, 100 um or less, 75 um or less, 50 um or less, 40 um or less, 30 um or less, 20 um or less, 10 um or less, 5 um or less, 4 um or less, 3 um or less, 2 um or less, 1.8 um or less, 1.5 um or less, 1 um or less, 0.5 um or less, 0.2 um or less, 0.1 um or less, 50 nm or less, 20 nm or less, 10 nm or less, or in a range between any of the two values, only the interference poor regions exist.

47. The apparatus of claim 1, wherein at least part of the sample is compressed into a thin layer that has an average thickness of in a range of 0.5-2 um, 0.5-3 um, 0.5-5 um, 0.5-10 um, 0.5-20 um, 0.5-30 um, or 0.5-50 um.

48. The apparatus of claim 1, wherein at least part of the sample is compressed into a thin layer that has an average thickness of 500 um or less.

49. The apparatus of claim 1, wherein at least part of the sample is compressed into a thin layer that has an average thickness of 200 um or less.

50. The apparatus of claim 1, wherein at least part of the sample is compressed into a thin layer that has an average thickness of 100 um or less.

51. The apparatus of claim 1, wherein at least part of the sample is compressed into a thin layer that has an average thickness of 50 um or less.

52. The apparatus of claim 1, wherein at least part of the sample is compressed into a thin layer that has an average thickness of 25 um or less.

53. The apparatus of claim 1, wherein at least part of the sample is compressed into a thin layer that has an average thickness of 10 um or less.

54. The apparatus of claim 1, wherein at least part of the sample is compressed into a thin layer that has an average thickness of 5 um or less.

55. The apparatus of claim 1, wherein at least part of the sample is compressed into a thin layer that has an average thickness of 3 um or less.

56. The apparatus of claim 1, wherein at least part of the sample is compressed into a thin layer that has an average thickness of 2 um or less.

57. The apparatus of claim 1, wherein at least part of the sample is compressed into a thin layer that has an average thickness of 1 um or less.

58. The apparatus of claim 1, wherein at least part of the sample is compressed into a thin layer that has an average thickness of 500 nm or less.

59. The apparatus of claim 1, wherein at least part of the sample is compressed into a thin layer that has an average thickness of 100 nm or less.

60. The apparatus of claim 1, wherein at least part of the sample is compressed into a thin layer that has an average thickness in the range of 0.5-2 um, 0.5-3 um, or 0.5-5 um.

61. The apparatus of claim 4, wherein the average thickness of the layer of uniform thickness is in the range of 2 um to 2.2 um and the sample is blood.

62. The apparatus of claim 4, wherein the average thickness of the layer of uniform thickness is in the range of 2.2 um to 2.6 um and the sample is blood.

63. The apparatus of claim 4, wherein the average thickness of the layer of uniform thickness is in the range of 1.8 um to 2 um and the sample is blood.

64. The apparatus of claim 4, wherein the average thickness of the layer of uniform thickness is in the range of 2.6 um to 3.8 um and the sample is blood.

65. The apparatus of claim 4, wherein the average thickness of the layer of uniform thickness is in the range of 1.8 um to 3.8 um and the sample is whole blood without a dilution by another liquid.

66. The apparatus of claim 4, wherein the average thickness of the layer of uniform thickness is about equal to a minimum dimension of an analyte in the sample.

67. The apparatus of claim 1, wherein the final sample thickness device is configured to analyze the sample in 300 seconds or less.

68. The apparatus of claim 1, wherein the final sample thickness device is configured to analyze the sample in 180 seconds or less.

69. The apparatus of claim 1, wherein the final sample thickness device is configured to analyze the sample in 60 seconds or less.

70. The apparatus of claim 1, wherein the final sample thickness device is configured to analyze the sample in 30 seconds or less.

71. The apparatus of claim 1, wherein the sample is original, diluted, or processed forms of: bodily fluids, stool, amniotic fluid, aqueous humour, vitreous humour, blood, whole blood, fractionated blood, plasma, serum, breast milk, cerebrospinal fluid, cerumen, chyle, chime, endolymph, perilymph, feces, gastric acid, gastric juice, lymph, mucus, nasal drainage, phlegm, pericardial fluid, peritoneal fluid, pleural fluid, pus, rheum, saliva, sebum, semen, sputum, sweat, synovial fluid, tears, vomit, urine, or exhaled breath condensate.

72. The apparatus of claim 1, wherein the sample is original, diluted, or processed forms of blood.

73. The apparatus of claim 1, wherein the sample comprises whole blood.

74. The apparatus of claim 1, wherein the sample comprises an aggregation agent that induces aggregation of the interference elements.

75. The apparatus of claim 1, wherein the analyte is a biomarker, an environmental marker, or a foodstuff marker.

76. The apparatus of claim 1, wherein the analyte is a biomarker indicative of the presence or severity of a disease or condition.

77. The apparatus of claim 1, wherein the analyte is a cell, a protein, or a nucleic acid.

78. The apparatus of claim 1, wherein the analyte comprises proteins, peptides, nucleic acids, synthetic compounds, inorganic compounds, organic compounds, bacteria, virus, cells, tissues, nanoparticles, and other molecules, compounds, mixtures and substances thereof.

79. The apparatus of claim 1, wherein the analyte is selected from Table B1, B2, B3 or B7 of PCT Application No. PCT/US2016/054,025.

80. The apparatus of claim 1, wherein the interference elements generate signals that interfere with the signal from the analyte.

81. The apparatus of claim 1, wherein the interference elements comprise: cells, tissues, or chemical or biological molecules.

82. The apparatus of claim 1, wherein the sample comprises blood interference elements comprise blood cells.

83. The apparatus of claim 1, wherein the sample comprises blood interference elements comprise red blood cells.

84. The apparatus of claim 1, wherein the sample comprises whole blood interference elements comprise red blood cells.

85. The apparatus of claim 1, wherein the sample holder comprises wells that configured to hold the sample.

86. The apparatus of claim 1, wherein the sample holder comprises a first plate, and a second plate, and spacers.

87. The apparatus of claim 1, wherein the sample holder comprises a first plate, a second plate, and spacers, wherein the spacers are configured to regulate a gap between the plates when the plates are pressed against each, compressing the sample into a thin layer.

88. The apparatus of claim 1, wherein the sample holder comprises a first plate, a second plate, and spacers, and wherein:
i. the plates are moveable relative to each other into different configurations, including an open configuration and a closed configuration;
ii. in the open configuration: the two plates are separated apart, the spacing between the plates is not regulated by the spacers, and the sample is deposited on one or both of the plates; and
iii. in the closed configuration, which is configured after the sample deposition in the open configuration: at least part of the sample is compressed by the two plates into a layer of highly uniform thickness and is substantially stagnant relative to the plates, wherein the uniform thickness of the layer is regulated by the plates and the spacers.

89. The apparatus of claim 1, wherein the sample holder comprises a Q-card, which comprises a first plate, a second plate, and spacers, wherein the spacers are configured to regulate a gap between the plates when the plates are pressed against each, compressing the sample into a thin layer.

90. The apparatus of claim 1, wherein
i. the sample holder comprises a first plate, a second plate, and spacers, wherein the spacers have a uniform height and a constant inter-spacer distance; and
ii. the sample is compressed by the sample holder into a thin layer with a uniform thickness that is regulated by the height of the spacers.

91. The apparatus of claim 1, wherein the sample is compressed into a layer of uniform thickness that substantially equals uniform height of spacers that are fixed to one or both of the plates.

92. The apparatus of claim 1, wherein the sample is compressed into a layer of uniform thickness that has a variation of less than 15%, 10%, 5%, 2%, 1%, or in a range between any of the two values.

93. The apparatus of claim 1, wherein the sample, when compressed, has a thickness of 500 nm or less, 1000 nm or less, 2 μm (micron) or less, 5 μm or less, 10 μm or less, 20 μm or less, 50 μm or less, 100 μm or less, 150 μm or less, 200 μm or less, 300 μm or less, 500 μm or less, 800 μm or less, 1 mm (millimeter) or less, 2 mm or less, 3 mm or less, 5 mm or less, 10 mm or less, or in a range between any two of these values.

94. The apparatus of claim 1, wherein the sample holder comprises a first plate and a second plate, wherein each of the plate has a thickness of 500 nm or less, 1000 nm or less, 2 μm (micron) or less, 5 μm or less, 10 μm or less, 20 μm or less, 50 μm or less, 100 μm or less, 150 μm or less, 200 μm or less, 300 μm or less, 500 μm or less, 800 μm or less, 1 mm (millimeter) or less, 2 mm or less, 3 mm or less, 5 mm or less, 10 mm or less, or in a range between any two of these values.

95. The apparatus of claim 74, wherein the aggregation agent induces aggregation of the interference elements.

96. The apparatus of claim 1, wherein the sample comprises blood and an aggregation agent that induces aggregation of red blood cells.

97. The apparatus of claim 96, wherein the aggregation agent comprises: fibrinogen (and subunits thereof), thrombin and prothrombin, certain dextran fractions (e.g. Dx-500, Dx-100, and Dx-70), poly(ethylene glycol), or polyvinylprrolidone (PVP, e.g. PVP-360 and PVP-40), or any combination thereof.

98. The apparatus of claim 96, wherein the aggregation agent is configured to induce the aggregation of at least 50%, 60%, 70%, 80%, 90%, or 95% of the red blood cells in the sample within 1, 2, 5, 10, 20, 30, or 60 minutes, or in a time range between any of the two values.

99. The apparatus of claim 1, wherein the imager comprises a camera.

100. The apparatus of claim 1, wherein the imager is a part of the detector.

101. The apparatus of claim 1, wherein the imager is the entirety of the detector.

102. The apparatus of claim 1, wherein the imager is directed by the software to capture one or more images of the sample, identify the interference element regions and the interference element free regions, and digitally separate the interference element regions from the interference element free regions.

103. The apparatus of claim 1, wherein the imager comprises a filter that is configured to filter signals from the sample.

104. The apparatus of claim 1, wherein the imager comprises a light source that is configured to illuminate the sample.

105. The apparatus of claim 1, wherein the detector is a mobile device.

106. The apparatus of claim 1, wherein the detector is a smart phone.

107. The apparatus of claim 1, wherein the detector is a smart phone and the imager is a camera as part of the smart phone.

108. The apparatus of claim 1, wherein the detector comprises a display that is configured to show the presence and/or amount of the analyte.

109. The apparatus of claim 1, wherein the detector is configured to transmit detection results to a third party.

110. The apparatus of claim 1, wherein the software is stored in a storage unit, which is part of the detector.

111. The apparatus of claim 1, wherein the software is configured to direct the detector to display the presence and/or amount of the analyte.

112. The apparatus of claim 1, wherein the software is configured to direct the imager to calculate the combined signal of the analyte from the interference element free regions.

113. The apparatus of claim 1, wherein the software is configured to direct the imager to disregard the signal of the analyte from the interference element regions.

114. The apparatus of claim 1, wherein the software is configured to direct the imager to increase signal contrast of the signals from the interference element regions to the signals from the interference element free regions

115. The apparatus of claim 1, wherein the software is configured to direct the detector to calculate a ratio of the signal from the interference element regions to the interference element free regions.

116. The apparatus of claim 1, wherein the apparatus is used for detection of proteins, peptides, nucleic acids, synthetic compounds, inorganic compounds, organic compounds, bacteria, virus, cells, tissues, nanoparticles, and other molecules, compounds, mixtures and substances thereof.

117. The apparatus of claim 1, wherein the apparatus is used for diagnostics, management, and/or prevention of human diseases and conditions.

118. The apparatus of claim 1, wherein the apparatus is are used for diagnostics, management, and/or prevention of veterinary diseases and conditions, or for diagnostics, management, and/or prevention of plant diseases and conditions.

119. The apparatus of claim 1, wherein the apparatus is used for environments testing and decontamination.

120. The apparatus of claim 1, wherein the apparatus is, wherein the apparatus or method are used for agricultural or veterinary applications.

121. The apparatus of claim 1, wherein the apparatus is used for food testing.

122. The apparatus of claim 1, wherein the apparatus is are used for drug testing and prevention.

123. The apparatus of claim 1, wherein the apparatus is used for detecting and/or measuring an analyte in blood.

124. The apparatus of claim 1, wherein the apparatus is used for a colorimetric assay.

125. The apparatus of claim 1, wherein the apparatus is used for a fluorescence assay.

126. The apparatus of claim 1, wherein the signal related to the analyte is an electrical signal or an optical signal.

127. The apparatus of claim 1, wherein the signal related to the analyte is an optical signal that allows the imager to capture images of the interference element rich region and the interference element poor region.

128. The apparatus of claim 1, wherein the signal related to the analyte is from a colorimetric reaction.

129. The apparatus of claim 1, wherein the signal related to the analyte is produced by illuminating the sample with an illumination source.

130. The apparatus of claim 2, wherein the plates are movable relative to each.

131. The apparatus of claim 4, wherein the spacers are fixed on one or both of the plates and have a uniform height.

132. The apparatus of claim 3, wherein the first plate and second plate are configured to compress the sample into a layer of uniform thickness that substantially equals the height of the spacers.

133. The apparatus of claim 4, wherein the spacers have a uniform height of 1 mm or less, 500 um or less, 400 um or less, 300 um or less, 200 um or less, 175 um or less, 150 um or less, 125 um or less, 100 um or less, 75 um or less, 50 um or less, 40 um or less, 30 um or less, 20 um or less, 10 um or less, 5 um or less, 4 um or less, 3 um or less, 2 um or less, 1.8 um or less, 1.5 um or less, 1 um or less, 0.5 um or less, 0.2 um or less, 0.1 um or less, 50 nm or less, 20 nm or less, 10 nm or less, or in a range between any of the two values.

134. The apparatus of claim 4, wherein the spacers have a uniform height in the range of 0.5-2 um, 0.5-3 um, 0.5-5 um, 0.5-10 um, 0.5-20 um, 0.5-30 um, or 0.5-50 um.

135. The apparatus of claim 2, wherein at least one of the plates has a thickness of 100 mm or less, 50 mm or less, 25 mm or less, 10 mm or less, 5 mm or less, 1 mm or less, 500 um or less, 400 um or less, 300 um or less, 200 um or less, 175 um or less, 150 um or less, 125 um or less, 100 um or less, 75 um or less, 50 um or less, 40 um or less, 30 um or less, 20 um or less, 10 um or less, 5 um or less, 4 um or less, 3 um or less, 2 um or less, 1.8 um or less, 1.5 um or less, 1 um or less, 0.5 um or less, 0.2 um or less, or 0.1 um or less, or in a range between any of the two values.

136. The apparatus of claim 2, wherein at least one of the plates has a thickness in the range of 0.5 to 1.5 mm; around 1 mm; in the range of 0.15 to 0.2 mm; or around 0.175 mm.

137. The apparatus of claim 2, wherein at least one of the plates has a lateral area of 1 mm² or less, 10 mm² or less, 25 mm² or less, 50 mm² or less, 75 mm² or less, 1 cm² (square centimeter) or less, 2 cm² or less, 3 cm² or less, 4 cm² or less, 5 cm² or less, 10 cm² or less, 100 cm² or less, 500 cm² or less, 1000 cm² or less, 5000 cm² or less, 10,000 cm² or less, 10,000 cm² or less, or in a range between any two of these values

138. The apparatus of claim 2, wherein at least one of the plates has a lateral area of in the range of 500 to 1000 mm²; or around 750 mm²

139. The apparatus of claim 4, wherein the Young's modulus of the spacers times the filling factor of the spacers is equal or larger than 10 MPa, wherein the filling factor is the ratio of the spacer area in contact with the layer of uniform thickness to the total plate area in contact with the layer of uniform thickness.

140. The apparatus of claim 2, wherein the thickness of a plate times the Young's modulus of the plate is in the range 60 to 750 GPa-um.

141. The apparatus of claim 90, wherein for a plate, the fourth power of the inter-spacer-distance (ISD) divided by the thickness of the plate (h) and the Young's modulus (E) of the flexible plate, ISD⁴/(hE), is equal to or less than 10⁶ um³/GPa.

142. The apparatus of claim 2, wherein one or both plates comprises a location marker, either on a surface of or inside the plate, that provide information of a location of the plate.

143. The apparatus of claim 2, wherein one or both plates comprises a scale marker, either on a surface of or inside the plate, that provide information of a lateral dimension of a structure of the sample and/or the plate.

144. The apparatus of claim 2, wherein one or both plates comprises an image marker, either on a surface of or inside the plate, that assists an imaging of the sample.

145. The apparatus of claim 90, wherein the inter-spacer distance is in the range of 7 um to 50 um.

146. The apparatus of claim 90, wherein the inter-spacer distance is in the range of 50 um to 120 um.

147. The apparatus of claim 90, wherein the inter-spacer distance is in the range of 120 um to 200 um.

148. The apparatus of claim 4, wherein the spacers are pillars with a cross-sectional shape selected from round, polygonal, circular, square, rectangular, oval, elliptical, or any combination of the same.

149. The apparatus of claim 4, wherein the spacers have a pillar shape and have a substantially flat top surface, wherein, for each spacer, the ratio of the lateral dimension of the spacer to its height is at least 1.

150. The apparatus of claim 4, wherein each spacer has the ratio of the lateral dimension of the spacer to its height is at least 1.

151. The apparatus of claim 4, wherein the minimum lateral dimension of spacer is less than or substantially equal to the minimum dimension of an analyte in the sample.

152. The apparatus of claim 1, wherein the minimum lateral dimension of spacer is in the range of 0.5 um to 100 um.

153. The apparatus of claim 1, wherein the minimum lateral dimension of spacer is in the range of 0.5 um to 10 um.

154. The apparatus of claim 1, wherein the spacers have a pillar shape, and the sidewall corners of the spacers have a round shape with a radius of curverture at least 1 um.

155. The apparatus of claim 1, wherein the spacers have a density of at least 100/mm².

156. The apparatus of claim 1, wherein the spacers have a density of at least 1000/mm².

157. The apparatus of claim 2, wherein at least one of the plates is transparent

158. The apparatus of claim 2, wherein at least one of the plates is made from a flexible polymer.

159. The apparatus of claim 4, wherein, for a pressure that compresses the plates, the spacers are not compressible and/or, independently, only one of the plates is flexible.

160. The apparatus of claim 2, wherein a plate has a thickness in the range of 10 um to 200 um.

161. The apparatus of claim 4, wherein the variation of thickness of the layer of uniform thickness is less than 30%.

162. The apparatus of claim 4, wherein the variation of thickness of the layer of uniform thickness is less than 10%.

163. The apparatus of claim 4, wherein the variation of thickness of the layer of uniform thickness is less than 5%.

164. The apparatus of claim 88, wherein the first and second plates are connected and are configured to be changed from the open configuration to the closed configuration by folding the plates.

165. The apparatus of claim 88, wherein the first and second plates are connected by a hinge and are configured to be changed from the open configuration to the closed configuration by folding the plates along the hinge.

166. The apparatus of claim 88, wherein the first and second plates are connected by a hinge that is a separate material to the plates, and are configured to be changed from the open configuration to the closed configuration by folding the plates along the hinge.

167. The apparatus of claim 88, wherein the first and second plates are made in a single piece of material and are configured to be changed from the open configuration to the closed configuration by folding the plates.

168. The apparatus of claim 4, wherein the layer of uniform thickness sample is uniform over a lateral area that is at least 1 mm².

169. The apparatus of claim 4, wherein the spacers are fixed on a plate by directly embossing the plate or injection molding of the plate.

170. The apparatus of claim 4, wherein the materials of the plate and the spacers are selected from polystyrene, PMMA, PC, COC, COP, or another plastic.

171. (canceled)

172. (canceled)

173. (canceled)

174. (canceled)

175. A non-transitory computer-readable medium comprising machine executable code that, upon execution by one or more computer processors, implements a method for detecting one or more analytes in a sample, the method comprising:
a. generating training data;
b. having a sample between two plates that has a spacing 200 um (micron) or less, wherein the sample comprising the one or more analytes and one or more interference elements, wherein the sample has a region in the sample that has less interference element concentration (“interference element poor region”) than another region in the sample (“interference element rich region”), and wherein one or both of the plates are configured to allow the at least a part of the sample visible through the one or both of the plates;
c. having an imager to image the sample;
d. in computer memory, generating a machine learning unit comprising one or more output calls for (i) the interference element poor region, and (ii) each of the one or more analytes in a sample, the sample comprising the one or more analytes and one or more interference elements, the sample at least partially contained within a sample holder that comprises a first plate and a second plate, wherein at least a part of the sample is between the first plate and second plate, and wherein one or both of the plates are configured to allow the at least a part of the sample visible through the one or both of the plates;
e. training the machine learning unit with a training set of samples, wherein the trained machine learning unit is configured to detect the one or more analytes from the sample of a subject using an imager and a detector,
wherein the sample comprises a mixture of analytes,
wherein the imager is configured to identify, in the at least a part of sample, a region that has less interference element concentration (“interference element poor region”) than another region in the sample layer (“interference element rich region”), and
wherein the detector is configured to detect a signal related to the analyte in an interference element poor region.

176. A method for detecting one or more analytes in a sample, the method comprising:
a. having a sample between two plates that has a spacing 200 um (micron) or less, wherein the sample comprising the one or more analytes and one or more interference elements, wherein the sample has a region in the sample that has less interference element concentration (“interference element poor region”) than another region in the sample (“interference element rich region”), and wherein one or both of the plates are configured to allow the at least a part of the sample visible through the one or both of the plates;
b. having an imager to image the sample;
c. generating training data;
d. in computer memory, generating a machine learning unit comprising one or more output calls, from the images, for (i) interference element poor region and (ii) each of the one or more analytes in a sample;
e. training the machine learning unit with a training set of samples, wherein the trained machine learning unit is configured to detect the one or more analytes from the sample of a subject using an imager and a detector,
wherein the sample comprises a mixture of analytes,
wherein the imager is configured to identify, in the at least a part of sample, a region that has less interference element concentration (“interference element poor region”) than another region in the sample layer (“interference element rich region”), and
wherein the detector is configured to detect a signal related to the analyte in an interference element poor region.

177. A system for detecting one or more analytes in a sample, the system comprising:
a. having a sample between two plates that has a spacing 200 um (micron) or less, wherein the sample comprising the one or more analytes and one or more interference elements, wherein the sample has a region in the sample that has less interference element concentration (“interference element poor region”) than another region in the sample (“interference element rich region”), and wherein one or both of the plates are configured to allow the at least a part of the sample visible through the one or both of the plates;
b. having an imager to image the sample;
c. computer memory for containing a machine learning unit to detect (i) the interference element poor region, and (ii) the one or more analytes in the sample, the sample comprising the one or more analytes and one or more interference elements, the sample at least partially contained within a sample holder that comprises a first plate and a second plate, wherein at least a part of the sample is between the first plate and second plate, and wherein one or both of the plates are configured to allow the at least a part of the sample visible through the one or both of the plates;
d. one or more computer processors that are individually or collectively programmed to:
i. generate training data;
ii. generate a machine learning unit comprising one or more output calls for each of the one or more analytes in a sample;
iii. train the machine learning unit with a training set of samples; and
iv. apply the machine learning unit to detect the one or more analytes from the sample of a subject, wherein the sample comprises a mixture of analytes;
e. an imager configured to identify, in the at least a part of sample, a region that has less interference element concentration (“interference element poor region”) than another region in the sample layer (“interference element rich region”); and
f. a detector configured to detect a signal related to the analyte in an interference element poor region.
